(19) United States
(12) Patent Application Publication
(10) Pub. No.: US 2005/0095646 A1
(43) Pub. Date: May 5, 2005

(54) Method of using a non-antibody protein to detect and measure an analyte

(76) Inventor: Michael I Sherman, Glen Ridge, NJ (US)

Correspondence Address: JONES DAY, 222 EAST 41ST ST, NEW YORK, NY, 10017, US

(21) Appl. No.: 10/495,239

(22) PCT Filed: Nov. 19, 2002
(86) PCT No.: PCT/US02/36959

Related U.S. Application Data
(60) Provisional application No. 60/331,706, filed on Nov. 19, 2001.

(51) Int. Cl. G01N 33/53

(52) U.S. Cl. 435/7.1

(57) ABSTRACT

The present invention relates to diagnostics, particularly binding assays for detecting and/or measuring an analyte. The present invention relates to methods for determining the presence and/or amount of an analyte by means of association with one or more non-antibody molecules, in particular non-antibody molecules derived from a species different from that of the analyte. Further, the present invention relates to methods for diagnosing and staging diseases by detecting and/or measuring analytes associated with certain diseases.

### RELATED APPLICATIONS

This application claims the benefit of application No. 60/331,706 filed Nov. 19, 2001, the entire disclosure of which is incorporated herein by reference in its entirety.

### 1. FIELD OF TIE INVENTION

The field of the invention is diagnostics, particularly binding assays for detecting and/or measuring an analyte. The present invention relates to methods for determining the presence and/or amount of an analyte by means of association with one or more non-antibody molecules, in particular molecules derived from a species different from that of the analyte. Further, the present invention relates to methods for diagnosing and staging diseases by detecting and/or measuring analytes associated with certain diseases.

### 2. BACKGROUND OF THE INVENTION

Methods for detecting an analyte in vitro are well known in the art. In general, the detection process requires contact with the analyte and a measurable report (qualitative or quantitative) that contact with the analyte has occurred. In the simplest of formats, the contact molecule and the reporter molecule can be on a single bimolecular molecule, but such assay formats tend to be less accurate than others in which more than one molecule is used in the detection process. More commonly, at least two different reagent molecules are used in diagnostic assays. For example, there can be a first molecule that binds to the analyte and a second molecule that records the successful binding event. Commonly, more than one molecule that can bind to the analyte is used in the procedure: a first molecule can be attached to a solid support to facilitate purification of the complex between the analyte and that first molecule. A second molecule that binds to the analyte can then be added. Such second molecule can provide a signal that binding between the first molecule and the analyte has occurred, or it can interact with a third molecule that transmits the signal.

Analyte detection methods typically are antibody-based immunoassays, assays using proteins from the same species as the analyte that interact with the analyte or polynucleotide-based hybridization screens. Immunoassays for detecting antigen analytes are well known in the art, and involve the formation of antigen-antibody complexes. The analyte may be added in liquid form, as is performed on immunodiffusion plates, or immobilized on a surface, as is performed using an enzyme-linked immunosorbent assay (“ELISA”) in the popular 96-well format. In an immunodiffusion assay, the antibody-antigen complex can be detected as a precipitation line. In a radioimmunoassay (“RIA”), a radioactive isotope is used to detect the presence of the analyte. In an enzyme immunoassay, a detectable marker produced by enzymatic activity (upon a chromogenic or fluorogenic substrate, for example) is used to detect the presence of the analyte (Engvall and Perlmann, 1972, “Enzyme-linked immunosorbent assay, Elisa. 3. Quantitation of specific antibodies by enzyme-labeled anti-immunoglobulin in antigen-coated tubes” J. Immunol. 109:129-35).

There are two classes of immunoassay. In the “direct” antibody immunoassay, an antibody that interacts with an analyte is measured directly, e.g., by RIA or ELISA. In this instance, the antibody acts both to contact the analyte and to provide a report of such interaction. In the “indirect” antibody immunoassay, a first antibody binds to the analyte and a second antibody, which binds to the first antibody, is detected and measured, e.g., by RIA or ELISA. Indirect immunoassays can also involve three antibodies: as an example, two of the antibodies can each bind the analyte (as is the case in the “sandwich” technique described below) and a third antibody, which binds to one of the other two antibodies, provides the report of a successful interaction. Indirect immunoassays are generally preferred over direct immunoassays because they tend to be more sensitive and specific and because the reporter antibody can be used as a generic reagent to measure many different antibodies, each of which binds to a different analyte.

One type of immunoassay is the “sandwich” technique. Sandwich assays commonly use an ELISA readout and involve the use of at least two antibodies. Typically, a sample potentially containing the analyte of interest is contacted with a first antibody on a solid support. After removing unbound sample, a second, enzyme-conjugated antibody is contacted with the analyte bound to the first antibody. After removal of unbound second antibody, a substrate (e.g. chromogenic or fluorogenic) of the enzyme is contacted with the antibody-analyte-antibody complex on the solid support. Production of a detectable marker indicates presence of the analyte in the sample, and the amount of detectable marker produced or the rate of production of a detectable marker can be used to determine the quantity of the analyte.

The sandwich assay is generally more sensitive and reliable than immunoassays in which only a single antibody is used to bind analyte because of reduced non-specific background production of the detectable marker. The sandwich assay in the example described above is a direct immunoassay because one of the antibodies that binds the analyte also acts as the reporter molecule, but sandwich assays can also be designed as indirect immunoassays if the second antibody in the example described above is not enzyme-conjugated but instead is detected by a third antibody that is enzyme-conjugated. Sandwich assays have been useful for diagnosing diseases as exemplified by the diagnosis of pseudorabies in swine using an ELISA-type assay (U.S. Pat. No. 4,562,147 to H. Joo).

As indicated in the foregoing, while the ELISA technique has proved successful in detecting an analyte of interest, the assay typically requires two antibodies specific for the analyte. Furthermore, when the sandwich technique is used, both antibodies must bind to the analyte but ideally they must not bind to the same part of the analyte or else one antibody will interfere with the binding of the other antibody to the analyte. In general, antibody specificity is difficult to engineer and generating two antibodies that differ in their site of binding to an analyte can be even more difficult to achieve. Supplies of such antibodies can be limited and production of the antibodies can be expensive and time-consuming. Moreover, antibodies of sufficient specificity and affinity can be particularly difficult to obtain when the target analyte is weakly antigenic. Obtaining two non-overlapping antibodies against weak antigens for sandwich assays is particularly challenging.

Therefore, there is a need in the art for methods of detecting and quantifying analytes in vitro that do not rely solely on antibodies for the binding and detection of target analytes.

### 3. SUMMARY OF THE INVENTION

The present invention relates to a method of using molecules to detect an analyte (i.e., molecule of interest being detected or measured in an analytical procedure), wherein at least one molecule is a non-antibody protein, and wherein at least one molecule is derived from a species different from that of the analyte. Preferably, the non-antibody binding protein is derived from a species different from that of the analyte.

Accordingly, in one embodiment, the present invention is a method for detecting or measuring an analyte comprising the steps of (a) contacting a first molecule that binds a biomolecular analyte with a sample containing the analyte under conditions that allow the analyte to be bound by the first molecule; (b) contacting the bound analyte with a second, different molecule that binds the analyte when the analyte is bound to the first molecule, under conditions that allow the analyte to be bound by the second molecule; (c) detecting or measuring binding of the second molecule to the analyte when the analyte is bound to the first molecule; wherein at least one of the first and second molecules is a non-antibody protein that is derived from a species different from that of the analyte; wherein the first molecule is attached to a solid support either before or after step (a); and wherein detection or measurement of binding indicates presence or amount, respectively, of the analyte.

In a further embodiment, the first and second molecules are non-antibody proteins that are derived from a species different from that of the analyte. In a particular embodiment, the method comprises, prior to step (a), the step of attaching the first molecule to the solid support.

In a further embodiment, the unbound sample is removed prior to step (c). In another further embodiment, the unbound first or second molecule is removed prior to step (c). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules that are not present in a complex comprising analyte, first molecule and second molecule.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, the present invention is a method for detecting or measuring an analyte comprising the steps of (a) contacting a first molecule that binds a biomolecular analyte with a sample containing the analyte under conditions that allow the analyte to be bound by the first molecule; (b) removing unbound sample; (c) contacting the bound analyte with a second, different molecule that binds the analyte when the analyte is bound to the first molecule, under conditions that allow the analyte to be bound by the second molecule; (d) removing unbound second molecule; and (e) detecting or measuring binding of the second molecule to the analyte when the analyte is bound to the first molecule, wherein at least one of the first and second molecules is a non-antibody protein that is derived from a species different from that of the analyte; wherein the first molecule is attached to a solid support either before or after step (a); and wherein detection or measurement of binding indicates presence or amount, respectively, of the analyte. In a further embodiment, the first and second molecules are non-antibody proteins that are derived from a species different from that of the analyte. In a particular embodiment, the method comprises, prior to step (a), the step of attaching the first molecule to the solid support.

All the different molecules are not required to bind to the analyte of interest. For example, to achieve signal amplification, a second, different molecule having a reporter enzyme conjugated thereto, can be used to bind a first molecule that is bound to the analyte. Further, a third, different molecule can bind to the second molecule. In one embodiment, several different secondary molecules that bind a first molecule that is bound to the analyte are used to amplify the signal corresponding to the presence of the analyte.

Accordingly, in one embodiment, the present invention is a method for detecting or measuring an analyte comprising the steps of (a) contacting a first molecule that binds a biomolecular analyte with a sample containing the analyte under conditions that allow the analyte to be bound by the first molecule; (b) contacting the bound, first molecule with a second, different molecule that binds the first molecule when the first molecule is bound to the analyte, under conditions that allow the second molecule to be bound by the first molecule; (c) detecting or measuring binding of the second molecule to the first molecule when the analyte is bound to the first molecule; wherein the first molecule is a non-antibody protein that is derived from a species different from that of the analyte; wherein the first molecule is attached to a solid support either before or after step (a); and wherein detection or measurement of binding indicates presence or amount, respectively, of the analyte.

In a further embodiment, the first and second molecules are derived from a species different from that of the analyte. In a specific further embodiment, the first and second molecules are non-antibody proteins. In a particular embodiment, the method comprises, prior to step (a), the step of attaching the first molecule to the solid support.

In a further embodiment, the unbound sample is removed prior to step (c). In another further embodiment, the unbound first or second molecule is removed prior to step (c). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules that are not present in a complex comprising analyte, first molecule and second molecule.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, the present invention is a method for detecting or measuring an analyte comprising the steps of (a) contacting a first molecule that binds a biomolecular analyte with a sample containing the analyte under conditions that allow the analyte to be bound by the first molecule; (b) removing unbound sample; (c) contacting the bound, first molecule with a second, different molecule that binds the first molecule when the first molecule is bound to the analyte, under conditions that allow the second molecule to be bound by the first molecule; (d) removing unbound second molecule; and (e) detecting or measuring binding of the second molecule to the first molecule when the analyte is bound to the first molecule; wherein the first molecule is a non-antibody protein that is derived from a species different from that of the analyte; wherein the first molecule is attached to a solid support either before or after step (a); and wherein detection or measurement of binding indicates presence or amount, respectively, of the analyte. In a further embodiment, the first and second molecules are derived from a species different from that of the analyte. In a specific further embodiment, the first and second molecules are non-antibody proteins. In a particular embodiment, the method comprises, prior to step (a), the step of attaching the first molecule to the solid support.

In one embodiment, one, two, three, four or five different molecules are used in an assay to detect and/or measure an analyte. In a further embodiment, two, three, four or five of the molecules are non-antibody proteins. In another embodiment, all different molecules are non-antibody proteins.

In one embodiment, at least one molecule that binds an analyte is derived from a species different from that of the analyte. In a further embodiment, all different molecules that bind the analyte are derived from a species different from that of the analyte. In a preferred embodiment, two non-antibody binding proteins, derived from a species different from that of an analyte of interest, are used in an assay to detect and/or measure the analyte.

In another embodiment, at least one molecule that binds an analyte is derived from a species different from that of another different molecule that binds the analyte, which species is different from that of the analyte. In another embodiment, at least one molecule that binds an analyte is derived from a species different from that of another different molecule that binds another molecule bound to the analyte, which species is different from that of the analyte. In yet another embodiment, all different molecules that bind an analyte are derived from the same species, which species is different from that of the analyte. In a specific further embodiment, first and second different molecules that bind the analyte are derived from the same species, which species is different from that of the analyte. In another specific embodiment, all different molecules that bind an analyte of interest are derived from yeast, and the analyte is derived from an organism other than yeast. In another specific embodiment, the analyte of interest is human-derived, and a first molecule that binds the analyte is derived from yeast. In yet another specific embodiment, the analyte of interest is human-derived, and one of the first or second molecules (that binds the analyte or that binds a first molecule when bound to the analyte) is derived from yeast.

In another embodiment, at least one molecule that binds an analyte of interest is derived from a species different from that of the analyte, and at least one of the molecules does not have a homolog in the species from which the analyte is derived. In another embodiment, all different molecules that bind an analyte of interest are derived from a species different from that of the analyte, and at least one of the molecules does not have a homolog in the species from which the analyte is derived. In a further embodiment, all of the molecules that bind an analyte of interest are derived from yeast, and the analyte is derived from an organism other than yeast, wherein at least one of the molecules does not have homolog in the species from which the analyte is derived.

A molecule that binds an analyte of interest can be an antibody or a non-antibody protein, wherein the protein is a full-length protein, a portion of a protein, or a peptide. In one embodiment, a first molecule that binds an analyte of interest is a non-antibody protein and a second different molecule that binds the first molecule bound to an analyte of interest is an antibody. In another embodiment, all different molecules that bind an analyte of interest are non-antibody proteins. In a specific embodiment, first and second molecules that bind an analyte of interest are non-antibody proteins. In another specific embodiment, first and second molecules that bind an analyte of interest are non-antibody proteins that are derived from a species different from that of the analyte and do not have a homolog in the species from which the analyte is derived.

A molecule that binds an analyte of interest can be unbound or bound to a solid support. In one embodiment, a molecule that binds an analyte of interest is unbound. In another embodiment, a molecule that binds an analyte of interest is bound to the surface of a solid support. In another embodiment, a molecule that binds an analyte of interest is bound to the surface of a well of the solid support. In a specific embodiment, a molecule that binds an analyte of interest is bound to the surface of a well of a-polystyrene, 96-well microtiter plate. In another embodiment, a molecule that binds an analyte of interest is bound to the surface of a well of a nanoarray device described in PCT International Publication No. WO 0183827 (published on Nov. 8, 2001) and in Zhu et al. (2000, “Analysis of yeast protein kinases using protein chips”, Nature Genet. 26:283-289).

A molecule that binds an analyte of interest or that binds a different molecule bound to the analyte can be conjugated to a detectable marker, or can be bound by a detectable marker. In one embodiment, a molecule that binds an analyte of interest is conjugated to a detectable marker such as, for example, fluorescein. In another embodiment, a molecule that binds an analyte of interest or that binds a molecule bound to the analyte is conjugated to an enzyme that produces a detectable marker such as, for example, alkaline phosphatase. In another embodiment, a molecule that binds an analyte of interest or that binds a different molecule bound to the analyte is conjugated to a hapten such as, for example, p-azobenzene arsonate. In another embodiment, a molecule that binds an analyte of interest or that binds a different molecule bound to the analyte is bound by a detectable marker such as, for example, a molecular mass marker.

Although at least one of the binding molecules used in the diagnostic assays described herein is a non-antibody molecule, one or more binding molecules used can be an antibody, preferably a monoclonal antibody. As a non-limiting example, a non-antibody binding protein that binds to an analyte can be identified by screening a protein array and a monoclonal antibody that binds to the non-antibody binding protein can serve as a reporter molecule by virtue of its conjugation to a detectable molecule.

Molecules useful for the methods of the present invention include, for example, proteins identified by any screening assay known in the art for detecting proteins of interest. One of ordinary skill in the art can recognize that many binding assays well known in the art can be used to identify and isolate molecules useful for the methods of the invention.

For example, binding proteins useful for the assays of the present invention can be identified by screening protein arrays with an analyte of interest. Accordingly, in one embodiment, a binding protein that binds an analyte of interest is identified by screening a protein array with the analyte. In a further embodiment, the protein array comprises at least one protein encoded by at least 50% or at least 70% of the known genes in a single species. In another further embodiment, the protein array comprises at least 50% of all proteins expressed in a single species (such that protein isoforms and splice variants are counted as a single protein). In another further embodiment, the protein array comprises at least 1000 proteins expressed in a single species. In yet another further embodiment, the protein array comprises proteins encoded by at least 1000 different known genes in a single species.

In a further embodiment, a first binding protein that binds an analyte of interest and is derived from a certain species, and a second binding protein derived from the same or a different species from which the first binding protein was derived are identified by screening a protein array. The analyte is then tested for the ability to bind the first binding protein in the presence of the second binding protein and to bind the second binding protein in the presence of the first binding protein. If the two binding proteins are capable of binding analyte in the presence of each other, such two binding proteins can then be used as reagents in a diagnostic assay analogous to the sandwich immunoassay.

In another further embodiment, a first binding protein that binds an analyte of interest and is derived from a certain species is obtained by screening a protein array. A complex comprising the first binding protein and the analyte is then screened on the same or different protein array used to identify the first binding protein to identify a second binding protein that binds to the complex. The second binding protein can then be tested against the separate components of the complex to determine whether the second binding protein binds to analyte or to the first binding protein. Second binding proteins can be characterized as reagents for various diagnostic assay formats in such a manner.

In yet another further embodiment, a first binding protein that binds an analyte of interest and is derived from a certain species is identified by screening a protein array and such first binding protein is used in a subsequent screening on a same or different array, containing at least one protein from the same species as that of the first binding protein or from a species different from that of the first binding protein, to identify a second binding protein that binds to the first binding protein. Such second binding protein can optionally be used in a subsequent screening on a same or different array, containing protein from the same species as that of the second binding protein or from a species different from that of the second binding protein, to identify a third binding protein that binds to the second binding protein, and so forth.

In another embodiment, the proteins among a population of proteins can be tested inter se to determine which proteins bind to which of every other protein in the population and the data obtained by such binding assays are documented as an “interaction profile.” The population of proteins can be large, and can encompass an entire proteome, for example. Once all, or almost all (e.g., greater than 50%, 60%, 70%, 80%, 90%, 95%), of the binding interactions among the population of proteins have been elucidated, an analyte of interest can be tested for binding to the protein population. For a first binding protein that is identified as binding to the analyte, reference to the interaction profile will indicate all second proteins that bind to such first binding proteins. Further reference to the interaction profile can then be used to indicate all third proteins that bind to such second binding protein, and so forth. This procedure for defining a series of binding proteins can be repeated for any binding protein that binds to the analyte.

Therefore, a database of such interactions can be useful for designing a diagnostic assay. For example, a human-derived analyte can be screened against a collection of proteins derived from a non-human species, which collection has been tested in binding assays inter se to identify which proteins in the collection bind to each other protein in the collection (i.e., an interaction profile). Proteins from the collection which bind the analyte are identified, and reference to interaction profile identifies other proteins in the collection that can be used as second-level or third-level binding proteins. In this manner, a binding assay for the analyte of interest can be designed when one binding protein (that binds the analyte) in the collection of proteins is known. Usually, the binding protein that binds the analyte can identified by performing only one screening assay. Such methods of designing a diagnostic assay are advantageous because the methods reduce the need to test and identify necessary binding proteins empirically for each different assay.

Screening a protein array with the analyte could identify one binding protein. Using the protein interaction database, second-, third-, and fourth-level binding proteins which bind to the first binding protein could be identified. In such an instance, the assay would not be a sandwich assay since there would be only one protein directly binding the analyte. If more than one protein is identified by screening the array with the analyte, then two proteins that bind the analyte simultaneously can be selected as the first-level binding proteins for a sandwich assay.

An analyte can be a member of a protein interaction database. In such an instance, the first binding protein could be identified by reference to the database. Screening of an array with the analyte would not be necessary since proteins that bind to the analyte would have already been determined using the protein interaction database. For example, a protein interaction database of the yeast proteome would contain all of the interactions between all yeast proteins. Therefore, if the analyte is a yeast protein, then all of the binding proteins would be included in the database. In such an instance, the analyte and the binding protein would be derived from the same species.

An analyte can be homologous to a member of a protein interaction database. All of the members of the database known to interact with a homolog of an analyte could be potential binders of the analyte. Therefore, it would be unnecessary to screen an array with the analyte. These potential binders could be individually tested for the ability to bind to the analyte. The second-, third-, and fourth-level proteins would all be known from the database.

A binding protein identified from a protein array as a binder for an analyte of interest can be a lipid binder. In such instance, a lipid could be used to bind to such a binding protein and direct or indirect detection of such lipid could be used, directly or indirectly, as an indicator of the presence of the analyte.

Alternatively, a binding protein identified from a protein array as a potential binder for an analyte of interest can be a nucleic acid binder. In such instance, a cognate nucleic acid could be used to bind to such binding protein and direct or indirect detection of such nucleic acid could be used as an indicator of the presence of the analyte. Thus, an analyte can first be bound by the nucleic acid, and a second binder (e.g., a protein from a species different from that of the analyte that recognizes the analyte-nucleic acid complex) can be bound and detected. Additionally, any of several alternative approaches to amplification of nucleic acids well known in the art can also be used to amplify the detection signal.

Accordingly, an analyte is preferably a biomolecule, and thus can be a protein, carbohydrate or lipid. An analyte can also be, without limitation, an intact cell or a component of the cell. However, an analyte can also be a small molecule (e.g., steroid, pharmaceutical drug). A small molecule is considered a non-peptide compound with a molecular weight of less than 500 daltons.

Other examples of analytes include, but are not limited to, bacteria, viruses, antigens, antibodies and polynucleotides. Particularly useful analytes are, for example, proteins, carbohydrates and lipids whose presence or levels correlate with a disease or disorder. The presence or levels of such analytes may correlate with the risk, onset, progression, amelioration and/or remission of a disease or disorder.

The detecting can be performed by, for example, autoradiography and/or phosphoimager analysis (for radioactivity), immunofluorescence (for fluorescently tagged ligands), immunochemistry (for antigenic ligands), mass spectrometry or atomic force microscopy (for molecular mass labels), infrared spectroscopy (for infrared labels), polymerase chain reaction (for amplifiable oligonucleotides), or colorimetric procedures (for reporter enzyme-linked ligands).

The present invention also relates to a method for determining a diagnosis or prognosis of a disease or disorder by assaying the presence or amount of an analyte that is correlated with a disease or disorder, and comparing the presence or amount of the analyte in an experimental sample with a control value, wherein a diagnosis or prognosis for a disease or disorder is determined when the presence or amount of analyte in the experimental sample differs from the control value.

Accordingly, in one embodiment, the present invention is a method of diagnosing a disease in a subject comprising the steps of (a) contacting a first molecule that binds an analyte of interest with a sample, suspected of containing the analyte, from the subject under conditions that allow the analyte to be bound by the first molecule; (b) contacting the bound analyte with a second, different molecule that binds the analyte when the analyte is bound to the first molecule, under conditions that allow the analyte to be bound by the second molecule; (c) detecting or measuring binding of the second molecule to the analyte when the analyte is bound to the first molecule, wherein detection or measurement of binding indicates presence or amount, respectively, of the analyte; wherein at least one of the first and second molecules is a non-antibody protein that is derived from a species different from that of the analyte; wherein the first molecule is attached to a solid support either before or after step (a); and wherein the disease is determined to be present when the presence or amount of analyte in step (c) differs from a control value representing the amount of analyte present in an analogous sample from a subject not having the disease or disorder.

In a further embodiment, the unbound sample is removed prior to step (c). In another further embodiment, the unbound first or second molecule is removed prior to step (c). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules that are not present in a complex comprising analyte, first molecule and second molecule.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, the present invention is a method of diagnosing a disease in a subject comprising the steps of (a) contacting a first molecule that binds an analyte of interest with a sample, suspected of containing the analyte, from the subject under conditions that allow the analyte to be bound by the first molecule; (b) removing unbound sample; (c) contacting the bound analyte with a second, different molecule that binds the analyte when the analyte is bound to the first molecule, under conditions that allow the analyte to be bound by the second molecule; (d) removing unbound second molecule; and (e) detecting or measuring binding of the second molecule to the analyte when the analyte is bound to the first molecule, wherein detection or measurement of binding indicates presence or amount, respectively, of the analyte; wherein at least one of the first and second molecules is a non-antibody protein that is derived from a species different from that of the analyte; wherein the first molecule is attached to a solid support either before or after step (a); and wherein the disease is determined to be present when the presence or amount of analyte in step (e) differs from a control value representing the amount of analyte present in an analogous sample from a subject not having the disease or disorder.

In another embodiment, the present invention is a method of diagnosing a disease in a subject comprising the steps of (a) contacting a first molecule that binds an analyte of interest with a sample, suspected of containing the analyte, from the subject under conditions that allow the analyte to be bound by the first molecule; (b) contacting the bound, first molecule with a second, different molecule that binds the first molecule when the first molecule is bound to the analyte, under conditions that allow the first molecule to be bound by the second molecule; and (c) detecting or measuring binding of the second molecule to the first molecule when the analyte is bound to the first molecule, wherein detection or measurement of binding indicates presence or amount, respectively, of the analyte; wherein the first molecule is a non-antibody protein that is derived from a species different from that of the analyte; wherein the first molecule is attached to a solid support either before or after step (a); and wherein the disease is determined to be present when the presence or amount of analyte in step (c) differs from a control value representing the amount of analyte present in an analogous sample from a subject not having the disease or disorder.

In a further embodiment, the unbound sample is removed prior to step (c). In another further embodiment, the unbound first or second molecule is removed prior to step (c). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules that are not present in a complex comprising analyte, first molecule and second molecule.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, the present invention is a method of diagnosing a disease in a subject comprising the steps of (a) contacting a first molecule that binds an analyte of interest with a sample, suspected of containing the analyte, from the subject under conditions that allow the analyte to be bound by the first molecule; (b) removing unbound sample; (c) contacting the bound, first molecule with a second, different molecule that binds the first molecule when the first molecule is bound to the analyte, under conditions that allow the first molecule to be bound by the second molecule; (d) removing unbound second molecule; and (e) detecting or measuring binding of the second molecule to the first molecule when the analyte is bound to the first molecule, wherein detection or measurement of binding indicates presence or amount, respectively, of the analyte; wherein the first molecule is a non-antibody protein that is derived from a species different from that of the analyte; wherein the first molecule is attached to a solid support either before or after step (a); and wherein the disease is determined to be present when the presence or amount of analyte in step (e) differs from a control value representing the amount of analyte present in an analogous sample from a subject not having the disease or disorder.

In another embodiment, the present invention is a method for staging a disease in a subject comprising the steps of (a) contacting a first molecule that binds an analyte of interest with a sample, suspected of containing the analyte, from the subject under conditions that allow the analyte to be bound by the first molecule; (b) contacting the bound analyte with a second, different molecule that binds the analyte when the analyte is bound to the first molecule, under conditions that allow the analyte to be bound by the second molecule; (c) detecting or measuring binding of the second molecule to the analyte when the analyte is bound to the first molecule, wherein detection or measurement of binding indicates presence or amount, respectively, of the analyte, wherein at least one of the first and second molecules is a non-antibody protein that is derived from a species different from that of the analyte; wherein the first molecule is attached to a solid support either before or after step (a); and wherein the stage of the disease in a subject is determined when the presence or amount of analyte in step (c) is compared with the amount of analyte present in an analogous sample from a subject having a particular stage of the disease or disorder.

In a further embodiment, the unbound sample is removed prior to step (c). In another further embodiment, the unbound first or second molecule is removed prior to step (c). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules that are not present in a complex comprising analyte, first molecule and second molecule.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, the present invention is a method for staging a disease in a subject comprising the steps of (a) contacting a first molecule that binds an analyte of interest with a sample, suspected of containing the analyte, from the subject under conditions that allow the analyte to be bound by the first molecule; (b) removing the unbound sample; (c) contacting the bound analyte with a second, different molecule that binds the analyte when the analyte is bound to the first molecule, under conditions that allow the analyte to be bound by the second molecule; (d) removing the unbound second molecule; and (e) detecting or measuring binding of the second molecule to the analyte when the analyte is bound to the first molecule, wherein detection or measurement of binding indicates presence or amount, respectively, of the analyte, wherein at least one of the first and second molecules is a non-antibody protein that is derived from a species different from that of the analyte; wherein the first molecule is attached to a-solid support either before or after step (a); and wherein the stage of the disease in a subject is determined when the presence or amount of analyte in step (e) is compared with the amount of analyte present in an analogous sample from a subject having a particular stage of the disease or disorder.

In another embodiment, the present invention is a method for staging a disease in a subject comprising the steps of (a) contacting a first molecule that binds an analyte of interest with a sample, suspected of containing the analyte, from the subject under conditions that allow the analyte to be bound by the first molecule; (b) contacting the bound first molecule with a second, different molecule that binds the first molecule when the first molecule is bound to the analyte under conditions that allow the first molecule to be bound by the second molecule; and (c) detecting or measuring binding of the second molecule to the analyte when the analyte is bound to the first molecule, wherein detection or measurement of binding indicates presence or amount, respectively, of the analyte, wherein at least one of the first and second molecules is a non-antibody protein that is derived from a species different from that of the analyte; wherein the first molecule is attached to a solid support either before or after step (a); and wherein the stage of the disease in a subject is determined when the presence or amount of analyte in step (c) is compared with the amount of analyte present in an analogous sample from a subject having a particular stage of the disease or disorder.

In a further embodiment, the unbound sample is removed prior to step (c). In another further embodiment, the unbound first or second molecule is removed prior to step (c). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules that are not present in a complex comprising analyte, first molecule and second molecule.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, the present invention is a method for staging a disease in a subject comprising the steps of (a) contacting a first molecule that binds an analyte of interest with a sample, suspected of containing the analyte, from the subject under conditions that allow the analyte to be bound by the first molecule; (b) removing unbound sample; (c) contacting the bound first molecule with a second, different molecule that binds the first molecule when the first molecule is bound to the analyte under conditions that allow the first molecule to be bound by the second molecule; (d) removing unbound second molecule; and (e) detecting or measuring binding of the second molecule to the analyte when the analyte is bound to the first molecule, wherein detection or measurement of binding indicates presence or amount, respectively, of the analyte, wherein at least one of the first and second molecules is a non-antibody protein that is derived from a species different from that of the analyte; wherein the first molecule is attached to a solid support either before or after step (a); and wherein the stage of the disease in a subject is determined when the presence or amount of analyte in step (e) is compared with the amount of analyte present in an analogous sample from a subject having a particular stage of the disease or disorder.

Accordingly, the methods of the present invention are useful for determining a diagnosis or prognosis for a disease or disorder such as, for example, an allergy, hormonal disorder, autoimmune disease, cancer, gastrointestinal disease, blood disorder, genetic disorder, food-borne illness, heart disease, infectious disease, circulatory disease, metabolic disorder, neurodegenerative disorder or behavioral disorder.

The invention also relates to kits comprising one or more binding molecules and/or detection means for detecting binding of a molecule to an analyte. In one embodiment, a kit comprises (a) in a first container, a purified biomolecular analyte; (b) in a second container, a first molecule that binds the analyte; and (c) a solid support having a second, different molecule attached thereto, wherein the second molecule binds the analyte when the analyte is bound to the first molecule, and wherein at least one of the first or second molecules is a non-antibody protein derived from a species different from that of the analyte.

In another embodiment, a kit comprises (a) in a first container, a purified biomolecular analyte; (b) a solid support having a first molecule attached thereto, wherein the first molecule binds the analyte, and wherein the first molecule is a non-antibody protein derived from a species different from that of the analyte; and (c) in a second container, a second, different molecule that binds the first molecule when the first molecule is bound to the analyte.

The invention also relates to kits designed to identify appropriate binding proteins for particular analytes. The invention also relates to kits comprising protein arrays for identifying binding proteins, and/or reagents useful for detecting binding of a molecule to analyte.

### 4. BRIEF DESCRIPTION OF THE FIGURES

FIGS. 1A-1B. Schematic of binding between an analyte and one or more proteins derived from a species different from that of the analyte. In (A), a non-antibody protein, M1, derived from a certain species binds to an analyte, A, derived from a different species than that of M1. In (B), a non-antibody protein, M1, from a particular species and a second non-antibody protein, M2, from either the same species or a different species than that of Ml, both bind, to different sites, on an analyte, A, such analyte being from a species different from that of protein M1 and/or protein M2.

FIGS. 2A-2B. Schematic of an assay for detecting an analyte of interest using two different non-antibody proteins that bind the analyte. (A) The wells of a microtiter plate are coated with a first protein, M1. After washing away unbound M1, a sample containing an analyte of interest, A, is added to each experimental well, along with a second protein, M2, which binds the analyte at a site different from the M1 binding site. M2 is conjugated to an enzyme capable of producing a detectable signal, D (step 1). The sample is incubated with M1 and M2 under conditions that allow the analyte to bind both M1 and M2. Standards of known analyte concentration and/or negative controls can be processed in parallel in control wells, if necessary. After washing away excess, unbound M2, an enzyme substrate, S, that produces a detectable product, P, is added to each well, and the mixture is incubated for a sufficient time and under conditions suitable for enzymatic activity (step 2). A detectable product is produced above a threshold level when the analyte of interest is present. The detectable product can be visually observed or measured (e.g., using a scanner or spectrophotometer) to provide qualitative or quantitative results. The amount of analyte present in the sample can be determined by comparison to a predetermined standard value or a standard curve determined in parallel. (B) In an alternative assay format, only the sample having an analyte of interest (A) is added to each experimental well containing M1 (step 1) and a washing step is added to remove unbound sample before addition of the second binding molecule, M2 (step 2). Determination of analyte is then carried out (step 3) as in (A).

FIG. 3. Schematic of an assay for detecting an analyte of interest using two different non-antibody proteins that bind the analyte and a reporter protein that binds one of the two analyte-binding proteins. The wells of a microtiter plate are coated with a first protein, M1. After washing away excess, unbound MI, the following are added to the well: 1) a sample having an analyte of interest, A, 2) a second protein, M2, which binds the analyte at a site different from M1 and 3) a third protein M3, which binds to M2 at an epitope different from the binding epitope of M2 for the analyte and which is conjugated to an enzyme capable of producing a detectable signal, D. The sample is incubated with M1, M2 and M3 under conditions that allow the analyte to bind both M1 and M2 and that allow M3 to bind M2. Standards of known analyte concentration and/or negative controls can be processed in parallel in control wells, if necessary. After washing away excess, unbound M2 and M3, an enzyme substrate that produces a detectable product can be added to each well, and determination of analyte can then be carried out as in FIG. 2. M1, M2, and M3 can be added simultaneously or sequentially.

FIGS. 4A-4B. (A) Schematic of an assay for detecting an analyte of interest using two different non-antibody proteins that bind the analyte and two reporter proteins that bind one of the two analyte-binding proteins. The wells of a microtiter plate are coated with a first protein, M1. After washing away excess, unbound M1, the following are added to the well: 1) a sample having an analyte of interest, A, 2) a second protein, M2, which binds the analyte at a site different from M1 3) a third protein M3, which binds to M2 at an epitope different from the binding epitope of M2 for the analyte and which is conjugated to an enzyme capable of producing a detectable signal, D, and 4) a fourth protein, M4, which binds to M2 at an epitope different from the binding epitopes of M2 or M3 and which is also conjugated to an enzyme capable of producing a detectable signal, D. The sample is incubated with M1, M2, M3 and M4 under conditions that allow the analyte to bind both M1 and M2 and that allow M3 and M4 to bind M2. Standards of known analyte concentration and/or negative controls can be processed in parallel in control wells, if necessary. After washing away excess, unbound M2, M3 and M4, an enzyme substrate that produces a detectable product can be added to each well, and determination of analyte can then be carried out as in FIG. 2. (B) Schematic of an assay for detecting an analyte of interest using two different proteins that bind the analyte and two different reporter proteins. The format and procedure are the same as in (A), except that the fourth binding protein, M4′, binds M3 rather than M2.

FIGS. 5A-5C. Schematic of an assay for detecting an analyte of interest using two different non-antibody proteins that bind the analyte and three reporter proteins. The wells of a microtiter plate are coated with a first protein, M1. After washing away excess, unbound M1, a sample having an analyte of interest, A, is added to each experimental well, along with a second protein, M2, which binds the analyte at a site different from M1 and three proteins that are conjugated to an enzyme capable of producing a detectable signal, D. The sample is incubated with the five proteins under conditions that allow the analyte to bind both M1 and M2 and that allow the other three proteins to bind to their cognate binding sites. Standards of known analyte concentration and/or negative controls can be processed in parallel in control wells, if necessary. After washing away excess, unbound proteins, an enzyme substrate that produces a detectable product can be added to each well, and determination of analyte can then be carried out as in FIG. 2. The three formats in (A),(B) and (C) are non-limiting alternative approaches of different binding combinations: in (A), M1 and M2 bind analyte, M3 binds M2, M4′ binds M3 and M5 binds M2; in (B), M1 and M2 bind analyte, M3 binds M2, and M4′ and M5′ bind M3; in (C), M1 and M2 bind analyte, M3 binds M2, M4′ binds M3 and M5″ binds M4′.

FIG. 6. Schematic of an assay for detecting an analyte of interest using two different non-antibody proteins, one that binds the analyte and undergoes an allosteric change when in contact with the analyte, the other that binds the first binding protein following such allosteric change. The wells of a microtiter plate are coated with a first protein, M1, which is capable of undergoing an allosteric change when in contact with the analyte. After washing away excess, unbound M1, a sample having an analyte of interest, A, is added to each experimental well. After washing to remove unbound sample, a second protein, M2, which is conjugated to an enzyme capable of producing a detectable signal, D and which binds M1 at a site different from the analyte, but only when M1 has bound analyte, is added to each well. Standards of known analyte concentration and/or negative controls can be processed in parallel in control wells, if necessary. After washing away excess, unbound M2, an enzyme substrate, S, that produces a detectable product, P, is added to each well, and the mixture is incubated for a sufficient time and under conditions suitable for enzymatic activity and determination of analyte can then be carried out as in FIG. 2. A detectable product is produced above a threshold level when the analyte of interest is present. The amount of the analyte present in the sample can be determined by estimation against a predetermined standard value or a standard curve determined in parallel.

FIG. 7. Schematic of an assay for detecting an analyte of interest using a non-antibody protein that binds an analyte that binds a known ligand. The wells of a microtiter plate are coated with a ligand, L, of an analyte of interest. After washing away excess, unbound L, a sample having an analyte of interest, A, is added to each experimental well, along with a second protein, M1′, which binds the analyte at a site different from the site of binding of M1′ to L and which is conjugated to an enzyme capable of producing a detectable signal, D. Standards of known analyte concentration and/or negative controls can be processed in parallel in control wells, if necessary. After washing away excess, unbound M1′, an enzyme substrate, S, that produces a detectable product, P, is added to each well, and the mixture is incubated for a sufficient time and under conditions suitable for enzymatic activity and determination of analyte can then be carried out as in FIG. 2. A detectable product is produced above a threshold level when the analyte of interest is present. The amount of the analyte present in the sample can be determined by estimation against a predetermined standard value or a standard curve determined in parallel.

FIGS. 8A-8B. Results of probing yeast proteome microarrays with human and yeast ras proteins. The left panel (A) shows a portion of the scanned image from the yeast proteome microarray that was probed with the human ras protein. The right panel (B) shows a portion of the scanned image from the yeast proteome microarray that was probed with the yeast ras protein. Solid white boxes are drawn around pairs of spots representing a single protein that interacts specifically with the probe. A dashed white box is drawn around control spots. It can be seen in this figure that four proteins interact with both the human and yeast ras proteins. It should also be noted that one yeast protein (designated with a star in the left panel) only interacts specifically with the human protein. This yeast protein, therefore, can be used as an affinity reagent to specifically detect the human ras protein. See Example 2 for details.

### 5. DETAILED DESCRIPTION OF THE INVENTION

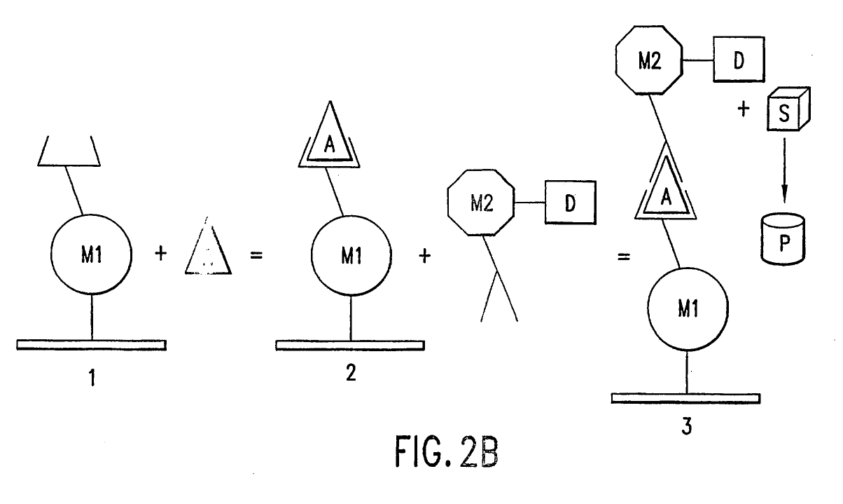

The present invention relates to a method for detecting an analyte of interest using non-antibody molecules that bind the analyte, and are derived from a species different from that of the analyte. An advantage of using molecules derived from a species different from that of the analyte is, inter alia, that less cross-reactivity is expected, thereby resulting in lower background levels, higher specificity and/or fewer false positives. Further advantage can be obtained by using such molecules that do not have homologous or orthologous gene products in the species from which the analyte is derived. Such molecules are likely to bind specifically to the analyte of interest and to no other compound in an experimental sample. Another advantage of using non-antibody molecules is that whereas the large majority of antibodies are directed against a particularly immunogenic epitope of an analyte, a binding protein might bind to any of a multiplicity of potential binding sites on an analyte without regard to immunogenicity of the analyte. This increases the possibility of identifying proteins that bind to different sites on an analyte and that can thus be used in tandem as reagents in a diagnostic assay, such as an assay analogous to a sandwich immunoassay, as schematically illustrated in FIG. 2.

Molecules that bind an analyte of interest, and therefore are useful for the methods of the present invention, can be identified and isolated by performing binding assays. For example, protein arrays can be screened using an analyte of interest as a probe, and binding of the analyte to proteins of the array can be detected and identified. Many other types of binding assays are known in the art, however, and are useful for the methods of the present invention.

Accordingly, the present invention contemplates the use of any binding assay useful for screening with an analyte of interest to identify molecules that bind the analyte. Many such assays are well known in the art, and the skilled artisan can appreciate that variants of such assays can be used in accordance with the present invention.

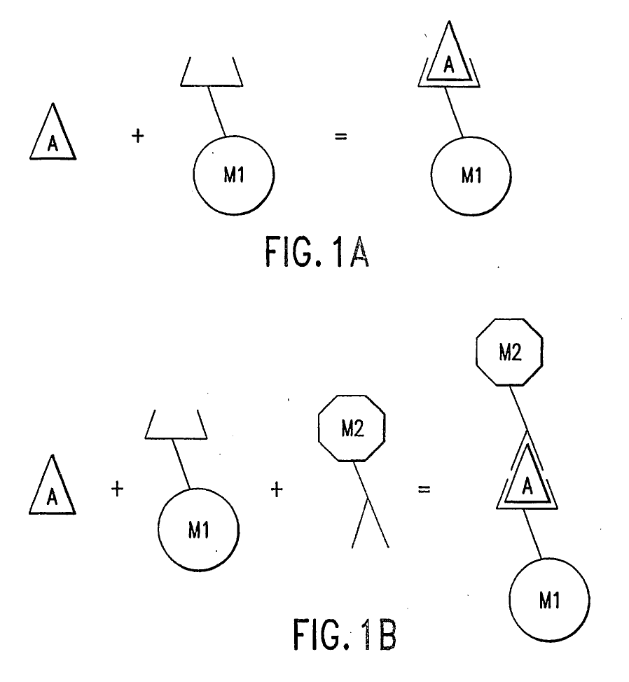

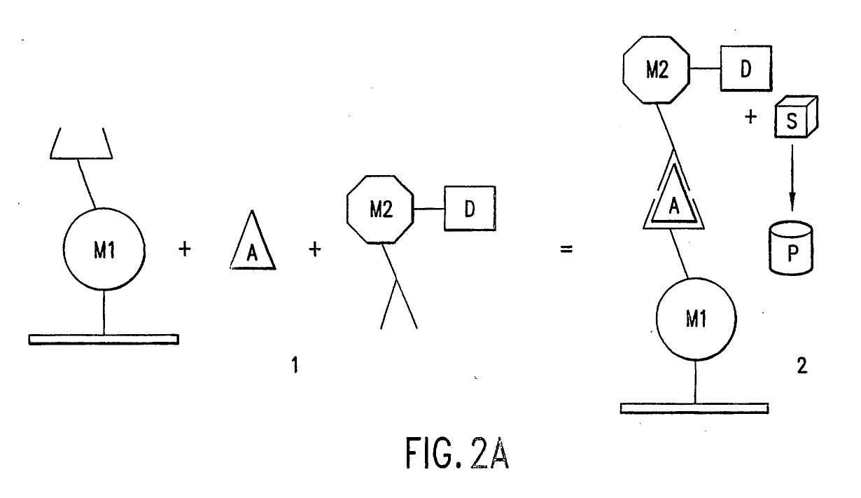

Binding assays can be performed one at a time to test sequentially the binding affinity of individual molecules with an analyte of interest, one such example of which is schematically presented in FIG. 1A. If more than one molecule is identified as a binder of an analyte of interest, the two molecules can be tested for the ability to bind the analyte simultaneously or sequentially (FIG. 1B).

It is believed likely that binding proteins can be found that bind to different domains on an analyte. One example of such an analyte is the EGF receptor. Binding of EGF to the extracellular domain of the EGF receptor allows the AP-2 clathrin adaptor complex to bind to the intracellular domain of the EGF receptor, particularly the micro 1 and micro 2 subunits of the adaptor complex (Sorkina et al., 2001, “Clatirin, adaptors and eps15 in endosomes containing activated epidermal growth factor receptors”, J. Cell Sci. 112:317-327). Another example of a protein that can bind two other proteins simultaneously is the Grb-2 protein, which binds simultaneously to the EGF receptor and to the protein, Sos (Zhang and Lautar, 1996, “A yeast three-hybrid method to clone ternary protein complex components”, Anal. Biochem. 242:68-72).

In another example, Zhu et al. (2001, “Global analysis of protein activities using proteome chips”, Science. 293:2101-2105) report that 39 proteins in their yeast protein array bound to calmodulin. It is unlikely that such a large number of proteins all bind to the same site on calmodulin and thus it is likely that there is a multiplicity of binding sites to which one or another of the thirty-nine identified binding proteins bind. FIG. 1B presents a schematic example of two binders, M1 and M2, that bind to distal sites on an analyte such that both molecules can bind simultaneously to the analyte. Such binding assays can be performed in solution and/or with a binding molecule or analyte bound to a solid support. The analyte can be contacted with binders simultaneously or sequentially.

Identification of proteins that can bind to an analyte while the analyte is bound to a first binding molecule would provide proteins that bind to different sites on the analyte and can bind simultaneously to the analyte. Accordingly, in one embodiment, a protein array is screened with an analyte, A, bound to a binding protein, M1. The proteins that bind to this complex could be used as first binding proteins in a diagnostic assay. Identifying multiple binding proteins in this manner would eliminate the need for re-screening binding proteins that were isolated individually for the ability to bind the analyte simultaneously.

Alternatively, an analyte of interest can be used to probe a collection of potential binding proteins. The collection of proteins is preferably arranged in an array to, inter alia, simplify the identification and isolation of proteins that bind the analyte. Accordingly, the present invention encompasses the use of protein arrays to identify proteins that bind an analyte of interest. Any array of proteins useful for screening with an analyte of interest to identify binding proteins can be used in accordance with the methods of the present invention. Such arrays can be any collection of proteins and can be from any source.

The use of protein microarrays (i.e., protein chips) for screening with an analyte of interest to identify proteins that bind the analyte has significant advantages over other approaches. One advantage of the protein microarray technology is that a large set of different proteins can be directly screened in a high-throughput manner. Furthermore, once the proteins to be placed on the array are prepared, protein array screening is inexpensive, amenable to automation, and the analysis of the screen is rapid using existing equipment and analytical software. Moreover, once identified, the clones encoding the protein(s) of interest (which have likely been inventoried in the process of preparing the protein arrays) can be amplified and expressed, and proteins that bind an analyte of interest can be produced quickly and inexpensively in large-scale quantities.

Another advantage of using a protein microarray is that a positionally addressable array provides a configuration such that each protein is at a known position on a solid support, thereby allowing each protein demonstrating binding to an analyte of interest to be identified from its position on the array. Thus, each protein on the array is preferably located at a known, predetermined position on the solid support such that each protein that binds the analyte can be identified from its position on the solid support.

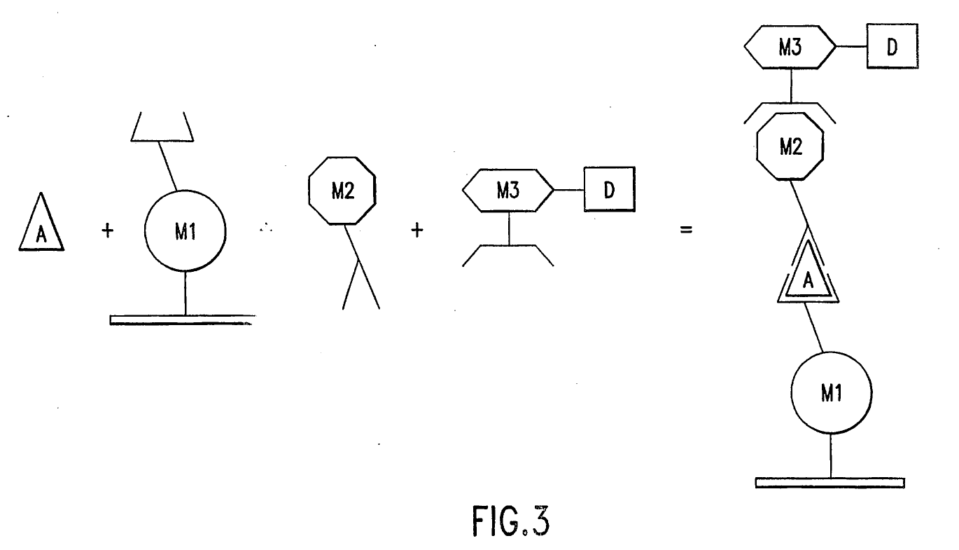

A further advantage of using a protein microarray is that an interaction profile for proteins in the array can be developed by testing such microarrays for binding activities with molecules other than the analyte. For example, if an analyte of interest is found to bind one of the 39 yeast proteins that has previously been determined to bind calmodulin (Zhu et al., 2001, “Global analysis of protein activities using proteome chips”, Science. 293:2101-2105), that binding protein can be used in a diagnostic assay for that analyte. For example, in the schematic example shown in FIG. 3, M1 and M2 are two proteins that bind to the analyte of interest and M2 is also a previously-identified calmodulin binding protein. In this format, provided that the analyte and calmodulin bind to non-overlapping sites on M2, calmodulin conjugated to an enzyme capable of producing a detectable signal, D, can serve as binding molecule M3 and can be used as a reporter molecule in the assay.

Molecules other than proteins can be identified as being useful reagents for diagnostic assays. For example, Zhu et al. (2001, “Global analysis of protein activities using proteome chips”, Science. 293:2101-2105) have reported that some 150 yeast proteins were found to bind to one or another of six phospholipids. Accordingly, if an analyte binds a binding protein in a microarray that has been documented to bind to a lipid, detection of a lipid could be used to measure binding to the analyte. In a non-limiting example, a sample of an analyte of interest can be added to a well-of a microtiter plate to which a first binding molecule for the analyte has been attached. After binding of analyte in the sample, the well can be washed and a second binding molecule that also binds a particular lipid can be added. After removing any unbound second binding molecule, the cognate lipid can be added to the well and the well washed once again. Measuring the presence of retained lipid would indicate the presence of analyte in the sample.

As another non-limiting example, Zhu et al. (2001, “Global analysis of protein activities using proteome chips”, Science. 293:2101-2105) have reported that a number of yeast proteins were identified to bind to nucleic acids. In a diagnostic format analogous to that described in the foregoing, with a lipid readout (detection of lipid determnines the presence of an analyte), an analogous assay can be designed in which nucleic acid is detected. Thus, if an analyte is bound by a binding protein in a microarray that has been documented to bind to a nucleic acid, a nucleic acid readout (detection of nucleic acid) could be used to measure binding to the analyte. In a non-limiting example, a sample of an analyte of interest can be added to a well of a microtiter plate in which a first binding molecule for the analyte has been attached to the surface of the well. After binding of analyte in the sample, the well can be washed and a second binding molecule that also binds a particular nucleic acid can be added. After removing any unbound second binding molecule, the cognate nucleic acid can be added to the well and the well can be washed once again. The retained nucleic acid can then be measured directly or, alternatively, prior to measurement of the nucleic acid, the nucleic acid can be amplified by any of a number of methods known in the art, including, but not limited to, polymerase chain reaction (Mullis, 1990, “Target amplification for DNA analysis by the polymerase chain reaction”, Ann. Biol. Clin. (Paris) 48(8):579-582; Ausubel et al., Current Protocols in Molecular Biology) or rolling circle amplification (Hatch et al., 1999, “Rolling circle amplification of DNA immobilized on solid surfaces and its application to multiplex mutation detection”, Genet. Anal. 15(2):35-40); Dean et al., 2001, “Rapid amplification of plasmid and phage DNA using phi29 DNA polymerase and multiply-primed rolling circle amplification”, Genome Res. 11:1095-1099; Schweitzer and Kingsmore, 2001, “Combining nucleic acid amplification and detection”, Curr. Opin. Biotech. 12:21-27).

A protein microarray can also be used to identify not only molecules that bind the analyte but also to identify a plurality of molecules that bind to the molecules that bind to the analyte. As a non-limiting example, by testing a population of proteins for all possible interactions inter se, an interaction profile can be documented for all of the proteins in the array. Such inter se testing can be achieved by extending the evaluation of biotinylated calmodulin (Zhu et al. 2001, “Global analysis of protein activities using proteome chips”, Science. 293:2101-2105) using the identified calmodulin binding proteins to screen an array of all other proteins against a protein array to all other proteins in the array to generate an interaction profile database. Such a database would contain all of the known interactions among a group of proteins. For example, a group of proteins comprising all or part of the yeast proteome could be used to prepare a protein array. Each protein member of the array could be used to evaluate its ability to bind to any of the other proteins in the array thereby identifying all of the interactions among all of the proteins in the array. All of these interactions are catalogued in an interaction profile database.

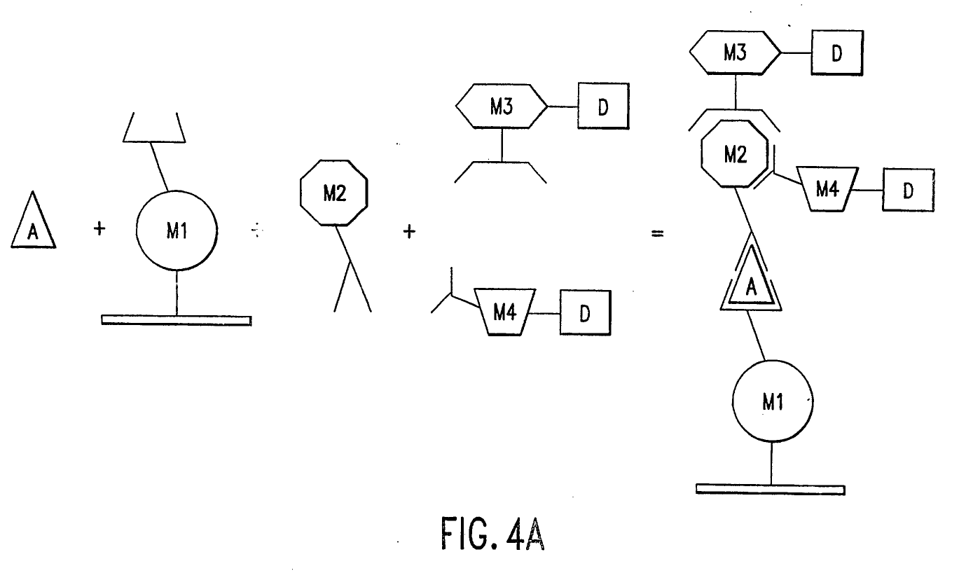

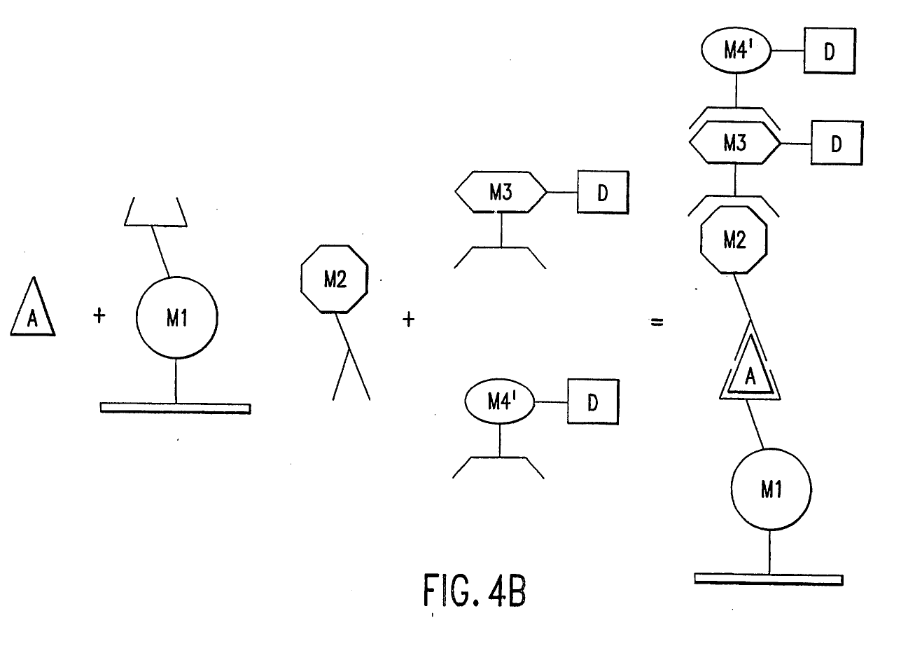

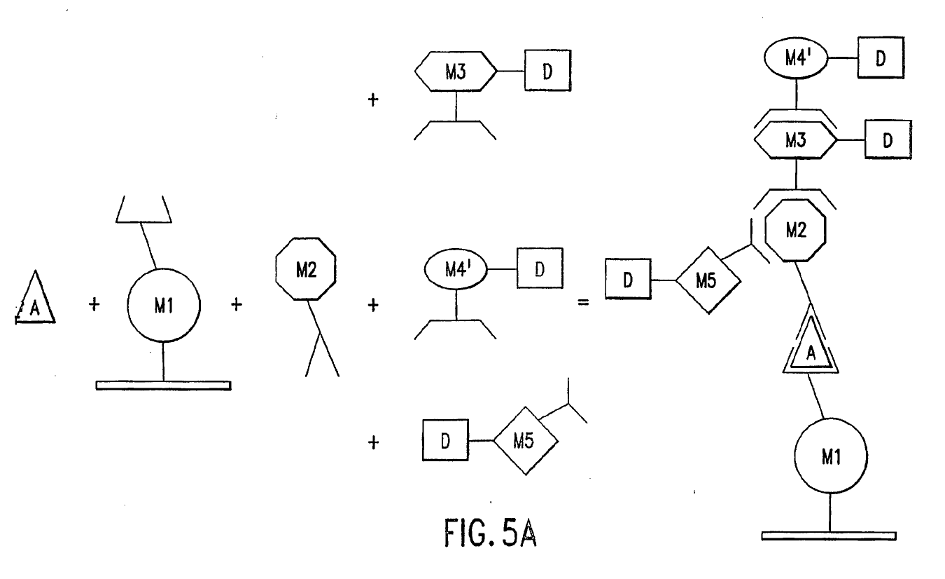

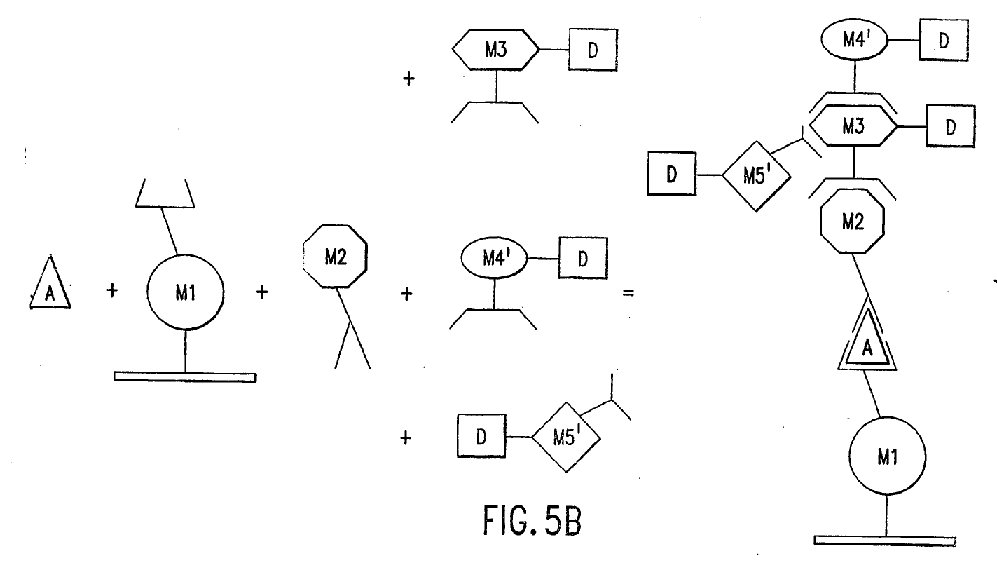

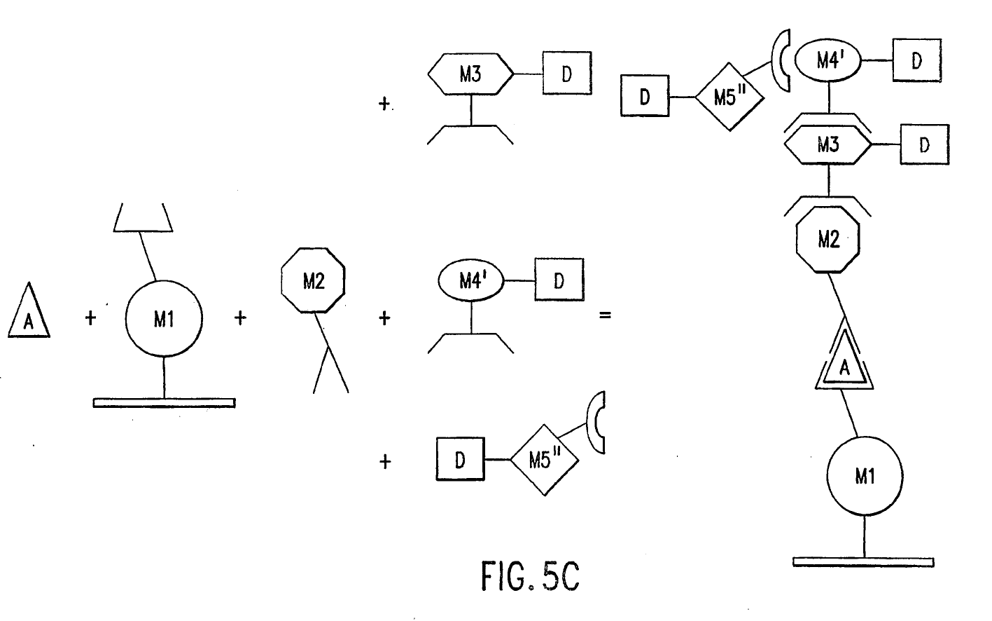

If an analyte of interest, for example, calmodulin, binds to a first binding protein in the array, then, by reference to the interaction profile database, an investigator would be able to conveniently predict not only proteins in the array that would be possible binders of the first binding protein (“second-level proteins”) but also proteins that could bind second level proteins (“third-level proteins”), proteins that could bind third-level proteins (“fourth-level proteins”) and so forth. Such second-level, third-level, fourth-level and so forth, binding proteins could be used as reagents in diagnostics assays formatted in various ways. FIGS. 4 and 5 illustrate non-limiting schematic examples of diagnostic assay formats utilizing four and five binding proteins, respectively. The format of the assay could also vary in the order in which the proteins are contacted with one another. Each binding protein of the assay could be contacted with the sample sequentially. Alternatively, the second and third or third and fourth-level binding proteins or all three proteins could be contacted with each other prior to contacting the sample.

The protein interaction database could also be used to identify binding proteins useful for a diagnostic assay in which the analyte is a small molecule. A small molecule could be used to screen a protein array to identify the first binding proteins. Once the first binding proteins are identified, a protein interaction database could be used to predict second, third and fourth level binding proteins. These predicted protein binders could be used in a diagnostic assay in which the analyte is a small molecule.

Although a predicted second-level protein from the foregoing example is a potentially useful reagent, it is also possible that the second level protein would bind to the same site, or to an overlapping site, on the first binding protein as does the analyte. In this instance, such a second-level protein might not be useful as a diagnostic reagent for detecting the analyte. A determination of potential usefulness of the second-level binding protein can be made, for example, by binding the analyte to the first binding protein and then screening an array of proteins with the analyte/first binding protein complex. If the second-level protein binds to a different site on the first binding protein from that of the analyte, the complex would be expected to bind to that second-level protein, but if the second-level protein binds to the same site on the first binding protein as the analyte, the complex would not be expected to bind to that protein. A comparison of results obtained from screening assays using analyte alone and using an analyte/binding-protein complex can distinguish overlapping and non-overlapping binders. Second-level, third-level, fourth-level, etc. proteins could be similarly tested for overlapping versus non-overlapping sites inter se and such information can be used with the interaction profile database to subsequently facilitate selection of useful diagnostic reagents.

Accordingly, arrays useful for identifying not only proteins that bind analytes of interest, but also second-level, third-level, fourth-level proteins (and so forth), include, without limitation, positionally addressable arrays comprising a plurality of proteins, with each protein being at a different position on a solid support. Examples of arrays useful for identifying and isolating binding proteins are described in PCT International Publication No. WO 0183827 to Yale University, published on Nov. 8, 2001, which is incorporated herein by reference in its entirety. For specific examples of protein arrays that can be used for screening, identifying and isolating binding proteins of interest, see Zhu et al. (2001, “Global analysis of protein activities using proteome chips”, Science. 293:2101-2105), Zhu and Snyder (2001, “Protein arrays and microarrays”, Curr Opin Chem Biol. 5:40-45) and Zhu et al. (2000, “Analysis of yeast protein kinases using protein chips”, Nature Genet. 26:283-289).

In one embodiment, the array comprises a plurality of proteins, wherein the plurality of proteins comprises at least one protein encoded by at least 50% or 70% of the known genes in a single species. In another embodiment, the array comprises a plurality of proteins, wherein the plurality of proteins comprises at least 50%, 60%, 70%, 80%, 90%, 95%, or 99% of all proteins expressed in a single species wherein protein isoforms and splice variants are counted as a single protein. In another embodiment, the array comprises a plurality of proteins, wherein the plurality of proteins comprises at least 1000, 1500, 2000, 2500, 3000, 4000, 5000, 6000, 7000, 8000, 9000, or 10000 proteins expressed in a single species. In yet another embodiment, the array comprises a plurality of proteins, wherein the plurality of proteins in aggregate comprise proteins encoded by at least 1000, 1500, 2000, 2500, 3000, 4000, 5000, 6000, 7000, 8000, 9000, or 10000 different known genes in a single species. In one particular embodiment, the species is a yeast. In another particular embodiment, the species is human. In another particular embodiment, the species is C. elegans. In another embodiment, the species is a bacterium such as Escherichia coli. In yet another embodiment, the species is a plant such as Arabidopsis thaliana.

Dense protein arrays can be produced such that assays for the presence and/or binding of proteins can be conducted in a high-throughput manner. For example, a protein chip can comprise a plurality of proteins that are printed on the surface of a solid support, wherein the density of printings is at least 100 printings/cm², 1000 printings/cm², 10,000 printings/cm², 100,000 printings/cm², 1,000,000 printings/cm², 10,000,000 printings/cm², 25,000,000 printings/cm², 10,000,000,000 printings/cm², or 100,000,000,000 printings/cm². Each individual protein sample on the chip constitutes a separate “printing.”

A protein chip can comprise a plurality of wells on the surface of a solid support, wherein the density of wells is at least 100 wells/cm², 1000 wells/cm², 10,000 wells/cm², 100,000 wells/cm², 1,000,000 wells/cm², 10,000,000 wells/cm², 25,000,000 wells/cm², 10,000,000,000 wells/cm², or 100,000,000,000 wells/cm². Variations of protein arrays comprising a plurality of printings or a plurality of wells can be useful for the methods of the present invention and are known in art such as, for example, in PCT International Publication No. WO 0183827 to Yale University, published on Nov. 8, 2001, which is incorporated herein by reference in its entirety.

The proteins on the array can be derived from a prokaryote or a eukaryote. Accordingly, the proteins on the array can be derived from a nematode, rodent, monkey, fruit fly, cow, horse, sheep, pig, chicken, turkey, quail, cat, dog, mouse, rat, rabbit, human, yeast, bacterium, plant or virus. For example, binding proteins derived from plants can be particularly useful for screening animal-derived (especially human-derived) samples for an analyte of interest. Such proteins can also comprise amino acids, natural or synthetic, that are useful for production, purification, binding, etc. of the protein, but are not naturally found in the native protein of interest. In such cases, despite the presence of amino acids (or other modifications) foreign to the native protein, the protein is considered derived from the species from which the native protein is derived.

Proteins on the arrays can include full-length proteins, chimeric proteins, portions of full-length proteins, and peptides (natural or synthetic), which can be prepared by, for example, recombinant overexpression, fragmentation of larger proteins, and/or chemical synthesis. Proteins can be overexpressed in cells derived from, for example, yeast, bacteria, insects, humans, or rodents. Further, a fusion protein comprising a defined domain attached to a natural or synthetic protein can be used.

Proteins can be embedded in artificial or natural membranes (e.g., liposomes, membrane fragments, membrane vesicles) prior to, or at the time of attachment to, the protein chip. Also, proteins can be attached to the solid support of the protein chip. Alternatively, the proteins can be delivered into wells of the protein chip, where they remain unbound to the solid support of the protein chip.

The solid support of a protein chip can comprise, for example, silicon, glass, quartz, polyimide, polymethylmethacrylate (Lucite), ceramic, amorphous silicon carbide, polystyrene, nitrocellulose, acrylamide, agarose, gold and/or any other material suitable for micro fabrication, microlithography or casting. See also PCT International Publication No. WO 0183827 to Yale University, published on Nov. 8, 2001, which is incorporated herein by reference in its entirety. In one embodiment, the solid support comprises a hydrophilic microtiter plate (e.g., Millipore™). In another embodiment, the solid support comprises a glass slide. In a specific embodiment, the solid support comprises a nickel-coated glass slide.

Each protein on the protein chip can be contacted with an analyte, and binding can be detected and quantified. Binding of analytes to proteins on the array can be detected by, for example, using radioactively labeled ligand followed by autoradiography and/or phosphoimager analysis; binding of enzyme or hapten (which is then detected using a fluorescently labeled or enzymatically labeled antibody, or by using high-affinity hapten ligand such as biotin or streptavidin); mass spectrometry; atomic force microscopy; surface plasmon resonance; fluorescent polarization methods; infrared-labeled compounds or proteins; amplifiable oligonucleotides, peptides or molecular mass labels; stimulation or inhibition of the protein's enzymatic activity; rolling circle amplification-detection; competitive PCR; calorimetric procedures; or biological assays (e.g., for virus titers).

Biotinylated analytes can be used to screen a protein array to aid in the detection of binding between an analyte and a binding protein. Weakly biotinylated proteins are more likely to maintain binding activity. Thus, a gentler biotinylation procedure is preferred so as to preserve the analyte's binding activity. Accordingly, in a particular embodiment, analytes are biotinylated to differing degrees using a biotin-transferring compound (e.g., Sulfo-NHS-LC-LC-Biotin; Pierce Catalog No. 21338, USA). The bound analyte can be identified by, for example, incubation with a fluorescently labeled avidin compound.

Alternatively, after incubation of proteins on a chip with an analyte, the bound analyte can be identified by, for example, mass spectrometry (Srinivas et al., 2001, “Proteomics in early detection of cancer”, Clin Chem. 47:1901-1911; Lakey et al., 1998, “Measuring protein-protein interactions”, Curr Opin Struct Biol. 8:119-123).

Thus, in one embodiment, a protein that binds an analyte of interest can be identified by (a) contacting the analyte with a positionally addressable array comprising a plurality of proteins, with each protein being at a different position on a solid support; and (b) detecting any analyte-protein interaction, wherein the plurality of proteins comprises at least one protein encoded by at least 50% or at least 70% of the known genes in a single species, and wherein detection of the interaction at a position on the solid support identifies a protein that binds the analyte.

In another embodiment, a protein that binds an analyte of interest can be identified by (a) contacting the analyte with a positionally addressable array comprising a plurality of proteins, with each protein being at a different position on a solid support; and (b) detecting any analyte-protein interaction wherein the plurality of proteins comprises at least 50% of all proteins expressed in a single species, wherein protein isoforms and splice variants are counted as a single protein and wherein detection of the interaction at a position on the solid support identifies a protein that binds the analyte.

In another embodiment, a protein that binds an analyte of interest can be identified by (a) contacting the analyte with a positionally addressable array comprising a plurality of proteins, with each protein being at a different position on a solid support; and (b) detecting any analyte-protein interaction wherein the plurality of proteins comprises at least 1000 proteins expressed in a single species and wherein detection of the interaction at a position on the solid support identifies a protein that binds the analyte.

In yet another embodiment, a protein that binds an analyte of interest can be identified by (a) contacting the analyte with a positionally addressable array comprising a plurality of proteins, with each protein being at a different position on a solid support; and (b) detecting any analyte-protein interaction wherein the plurality of proteins in aggregate comprise proteins encoded by at least 1000 different known genes in a single species and wherein detection of the interaction at a position on the solid support identifies a protein that binds the analyte.

Relatively low concentrations of molecules that bind an analyte of interest can be used in assays with high-affinity binding molecules. Moreover, use of high-affinity binding molecules can result in greater specificity and lower background signal. High-affinity binding proteins can be identified and isolated by conducting binding assays with an analyte under stringent conditions such as, for example, in the presence of detergent, and/or at a high or low pH and/or at low concentration of analyte or candidate binding molecule. Depending on the particular analyte and/or binding molecule, screening assays can be conducted in the presence of higher concentrations of detergent. The temperature and osmotic strength at which the screening assays are conducted can also be varied to influence the stringency.

Proteins that bind an analyte of interest identified by screening a protein array, for example, can be synthesized and isolated in a readily scalable format, amenable to high-throughput analysis. Such methods include, without limitation, synthesizing and purifying proteins in an array format that is compatible with automation technologies. For example, a method for synthesizing and isolating a protein that binds an analyte of interest can comprise the steps of growing a eukaryotic cell transformed with a vector having a heterologous sequence operatively linked to a regulatory sequence, contacting the regulatory sequence with an inducer that enhances expression of a protein encoded by the heterologous sequence, lysing the cell, contacting the protein with an agent such that a complex between the protein and agent is formed, isolating the complex from cellular debris, and isolating the protein from the complex, wherein each step is conducted in a 96-well format.

Any expression construct having an inducible promoter to drive protein synthesis can be used. Preferably, the expression construct is tailored to the cell type to be used for transformation. Any host cell that can be grown in culture can be used to synthesize a protein that binds an analyte of interest. Compatibility between expression constructs and host cells are known in the art. Host cells that can overproduce a protein and cause proper synthesis, folding, and post-translational modification of the protein are preferred. Preferably, such protein processing forms epitopes, binding sites, etc. useful for the assays of the invention. Accordingly, a eukaryotic cell (e.g., yeast, insect cell, human cell) is preferably used to synthesize eukaryotic proteins. Cells useful for expression of engineered proteins are known in the art, and variants of such cells and expression systems can be appreciated by one of ordinary skill in the art.

In one embodiment, a eukaryotic expression system is used. For example, the InsectSelect system (Invitrogen, Carlsbad, Calif.) simplifies the expression of high-quality proteins and eliminates the need to generate and amplify viral stocks. A preferred vector in this system is pIB/V5-His TOPO TA vector (catalog no. K890-20).

In another example, the BAC-TO-BAC™ system (Lifetech, Rockville, Md.) can be used. The BAC-TO-BAC™ system generates recombinant baculovirus by relying on site-specific transposition, rather than homologous recombination, in E. coli. Gene expression is driven by the highly active polyhedrin promoter, and therefore the protein of interest can represent up to 25% of the cellular protein in infected insect cells.

In another embodiment, a yeast expression system is used. In a particular further embodiment, a yeast expression system is used to overexpress yeast proteins that bind an analyte of interest.

Although proteins can be harvested from cells at any point in the cell cycle, cells are preferably isolated during logarithmic phase when protein synthesis is enhanced. For example, yeast cells can be harvested between OD₆₀₀=0.3 and OD₆₀₀=1.0, preferably between OD₆₀₀=0.5 and OD₆₀₀=0.9, more preferably between OD₆₀₀=0.6 and OD₆₀₀=0.8. In another example, yeast cells can be harvested at a density between 0.5×10⁶ to 1.0×10⁶ cells/ml, preferably 0.6×10⁶ to 0.9×10⁶ cells/ml, more preferably 0.7×10⁶ to 0.8×10⁶ cells/ml. In a particular embodiment, proteins are harvested from the cells at a point after mid-log phase.

In a specific embodiment, yeast cells are harvested at OD₆₀₀=0.3, OD₆₀₀=0.4, OD₆₀₀=0.5, OD₆₀₀=0.6, OD₆₀₀=0.7, OD₆₀₀=0.8 or OD₆₀₀=0.9. In another specific embodiment, yeast cells are harvested at a density of 0.5×10⁶ cells/ml, 0.6×10⁶ cells/ml, 0.7×10⁶ cells/ml, 0.8×10⁶ cells/ml, 0.9×10⁶ cells/ml or 1.0×10⁶ cells/ml. Harvested cells can be stored frozen for future manipulation.

The harvested cells can be lysed by a variety of methods known in the art. The method of lysis should be suited to the type of host cell. For example, a lysis buffer containing fresh protease inhibitors is added to yeast cells, along with an agent that disrupts the cell wall (e.g., sand, glass beads, zirconia beads), after which the mixture is shaken violently using a shaker (e.g., vortexer, paint shaker). The resulting cellular debris can be separated from the protein by, for example, centrifugation. Additionally, to increase purity of the protein sample in a high-throughput fashion, the protein-enriched supernatant can be filtered. The filter preferably comprises a solid support having low protein binding. In a preferred embodiment, a filter on a non-protein-binding solid support is used. Further, these steps can be repeated on the fraction containing the cellular debris to increase the yield of protein.

The proteins that bind an analyte of interest can then be purified from the protein-enriched supernatant using a variety of affinity purification methods known in the art. Affinity tags useful for affinity purification of fusion proteins include, but are not limited to, calmodulin, trypsin/anhydrotrypsin, glutathione, immunoglobulin domains, maltose, nickel, or biotin and its derivatives, which bind to calmodulin-binding protein, bovine pancreatic trypsin inhibitor, glutathione-S-transferase (“GST tag”), antigen or Protein A, maltose-binding protein, poly-histidine (“His tag”), and avidin/streptavidin, respectively. Fusion proteins comprising proteins that bind an analyte of interest, can be affinity purified using an appropriate binding compound and isolated by, for example, capturing the complex containing bound proteins on a low protein-binding filter. Placing one affinity tag on one end of the protein, and a second affinity tag on the other end of the protein can aid in purifying full-length proteins.

The purified proteins are preferably stored in a medium that stabilizes protein and prevents desiccation of the sample. For example, purified binding proteins can be stored in a liquid of high viscosity such as, for example, 15% to 50% glycerol, preferably in about 40% glycerol. In a specific embodiment, purified binding proteins are stored in a solution of 10%, 20%, 30%, 40%, 50%, 60% or 70% glycerol, preferably 15% to 55% glycerol; more preferably in 25% to 45% glycerol.

5.1. Method for Detecting an Analyte Using a Non-Antibody Molecule that Binds the Analyte

The present invention relates to an assay for detecting and/or measuring an analyte (i.e., molecule of interest being detected or measured in an analytical procedure) using molecules, wherein at least one molecule is a non-antibody protein, and wherein at least one molecule is derived from a species different from that of the analyte. The present invention contemplates the use of at least one molecule that binds an analyte or that binds another different molecule bound to the analyte, preferably 2, 3, 4, 5, 6, 7, 8, 9 or 10 different molecules, more preferably 2, 3, 4 or 5 different molecules, most preferably 2 or 3 different molecules. In a specific embodiment, two such molecules that are non-antibody proteins, and are derived from a species different from that of the analyte, are used to assay for the presence and/or concentration of the analyte in a sample.

In one embodiment, a non-limiting example of which is shown schematically in FIG. 2A, the present invention is a method for detecting or measuring an analyte comprising the steps of (a) contacting a first molecule that binds a biomolecular analyte with a sample containing the analyte under conditions that allow the analyte to be bound by the first molecule; (b) contacting the bound analyte with a second, different molecule that binds the analyte when the analyte is bound to the first molecule, under conditions that allow the analyte to be bound by the second molecule; (c) detecting or measuring binding of the second molecule to the analyte; wherein at least one of the first and second molecules is a non-antibody protein that is derived from a species different from that of the analyte; wherein the first molecule is attached to a solid support either before or after step (a); and wherein detection or measurement of binding indicates presence or amount, respectively, of the analyte. In a preferred embodiment, the first and second molecules are non-antibody proteins that are derived from a species different from that of the analyte. In a preferred embodiment, binding is detected or measured when the analyte is bound to the first molecule. In another preferred embodiment, binding is detected or measured when the analyte is bound, through the first molecule, to a solid support.

In a further embodiment, the unbound sample is removed prior to step (c). In another further embodiment, the unbound first or second molecule is removed prior to step (c). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules (e.g., contaminants, excess reagents) that are not present in a complex comprising analyte, first molecule and second molecule.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, a non-limiting example of which is shown schematically in FIG. 2B, the present invention is a method for detecting or measuring an analyte comprising the steps of (a) contacting a first molecule that binds a biomolecular analyte with a sample containing the analyte under conditions that allow the analyte to be bound by the first molecule; (b) removing unbound sample; (c) contacting the bound analyte with a second, different molecule that binds the analyte when the analyte is bound to the first molecule, under conditions that allow the analyte to be bound by the second molecule; (d) removing unbound second molecule; and (e) detecting or measuring binding of the second molecule to the analyte; wherein at least one of the first and second molecules is a non-antibody protein that is derived from a species different from that of the analyte; wherein the first molecule is attached to a solid support either before or after step (a); and wherein detection or measurement of binding indicates presence or amount, respectively, of the analyte. In a preferred embodiment, the first and second molecules are non-antibody proteins that are derived from a species different from that of the analyte. In a preferred embodiment, binding is detected or measured when the analyte is bound to the first molecule. In another preferred embodiment, binding is detected or measured when the analyte is bound, through the first molecule, to a solid support.

In an alternate embodiment, a step to remove unbound sample or binding molecule may be performed at any step prior to step (e). For example, several steps prior to step (e) can be introduced, in lieu of steps (b) and (d), to selectively remove unbound sample, unbound first molecules or unbound second molecules.

Each different molecule may bind to the analyte of interest. Alternatively, a second, different molecule can bind to a first molecule bound to the analyte. Further, in a non-limiting example, shown schematically in FIG. 3, a third, different molecule can bind to the second molecule. Further, in a non-limiting example shown schematically in FIG. 4B, a fourth, different molecule can bind to the third molecule. Further, in a non-limiting example, shown schematically in FIG. 5C, a fifth, different molecule can bind to the fourth molecule, and so on. In one embodiment, more than one different secondary molecule that binds a different molecule bound to an analyte are used to amplify the signal corresponding to the presence and/or amount of the analyte, as illustrated schematically in non-limiting examples in FIGS. 4A and 5A. In one further embodiment, more than one different secondary molecule binds different molecules that bind either to an analyte or to one or more different molecules that bind to the analyte are used to amplify the signal corresponding to the presence and/or amount of the analyte, as illustrated schematically in non-limiting examples in FIGS. 4B, 5B and 5C. The kind of “cascade” binding of molecules as described in the foregoing embodiments can be effective for detecting an analyte in low abundance by amplifying the signal many fold over signal obtained when using molecules that bind only to the analyte. The increase in sensitivity of the assay can be especially advantageous when the target analyte is present at a low concentration in the sample.

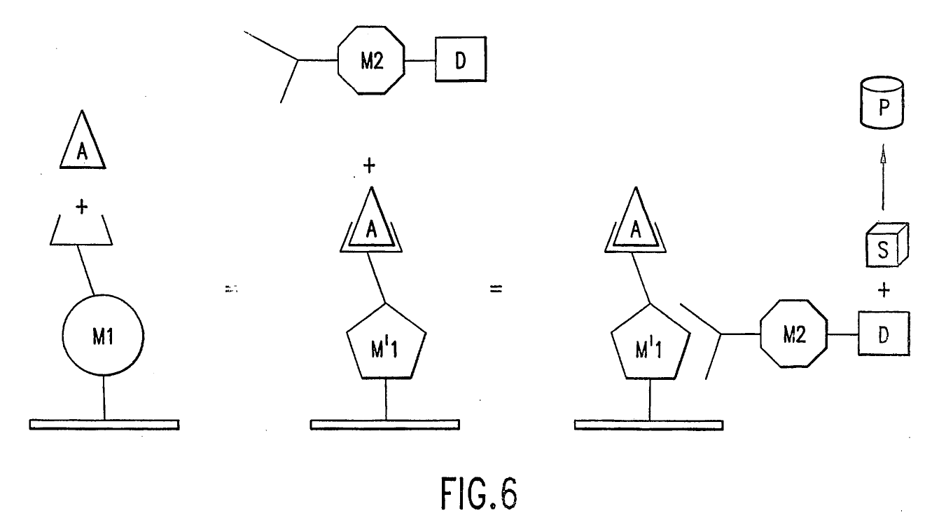

Many molecules exist in different conformations and some of these molecules assume certain conformations only when bound to a certain second molecule. Such allosteric changes are commonly found among proteins involved in signal transduction. The present invention can take advantage of such allosteric proteins as binding proteins. For example, in one such embodiment, a non-limiting example of which is shown in FIG. 6, the present invention is a method for detecting or measuring an analyte comprising the steps of (a) contacting a first molecule that binds a biomolecular analyte with a sample containing the analyte under conditions that allow the analyte to be bound by the first molecule; (b) contacting the bound, first molecule with a second, different molecule that binds the first molecule only when the first molecule is bound to the analyte, under conditions that allow the second molecule to be bound by the first molecule; and (c) detecting or measuring binding of the second molecule to the first molecule; wherein the first molecule is a non-antibody protein that is derived from a species different from that of the analyte; wherein the first molecule is attached to a solid support either before or after step (a); and wherein detection or measurement of binding indicates presence or amount, respectively, of the analyte.

In a preferred embodiment, binding is detected or measured when the analyte is bound to the first molecule. In another preferred embodiment, binding is detected or measured when the analyte is bound, through the first molecule, to a solid support.

In one embodiment, different second molecules, each of which binds to the first molecule, are used. In another embodiment, a third molecule that binds to a second molecule is used. In a preferred embodiment, the molecule that binds the analyte is a non-antibody protein that is derived from a species different from that of the analyte.

In a further embodiment, the unbound sample is removed prior to step (c). In another further embodiment, the unbound first or second molecule is removed prior to step (c). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules (e.g., contaminants, excess reagents) that are not present in a complex comprising analyte, first molecule and second molecule.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, the present invention is a method for detecting or measuring an analyte comprising the steps of (a) contacting a first molecule that binds a biomolecular analyte with a sample containing the analyte under conditions that allow the analyte to be bound by the first molecule; (b) removing unbound sample; (c) contacting the bound, first molecule with a second, different molecule that binds the first molecule only when the first molecule is bound to the analyte, under conditions that allow the second molecule to be bound by the first molecule; (d) removing unbound second molecule; and (e) detecting or measuring binding of the second molecule to the first molecule; wherein the first molecule is a non-antibody protein that is derived from a species different from that of the analyte; wherein the first molecule is attached to a solid support either before or after step (a); and wherein detection or measurement of binding indicates presence or amount, respectively, of the analyte.

In a preferred embodiment, binding is detected or measured when the analyte is bound to the first molecule. In another preferred embodiment, binding is detected or measured when the analyte is bound, through the first molecule, to a solid support.

In one embodiment, different second molecules, each of which binds to the first molecule, are used. In another embodiment, a third molecule that binds to the second molecule is used. In a preferred embodiment, the molecule that binds the analyte is a non-antibody protein that is derived from a species different from that of the analyte.

In an alternate embodiment, a step to remove unbound sample or binding molecule may be performed at any step prior to step (e). For example, several steps prior to step (e) can be introduced, in lieu of steps (b) and (d), to selectively remove unbound sample, unbound first molecules or unbound second molecules.

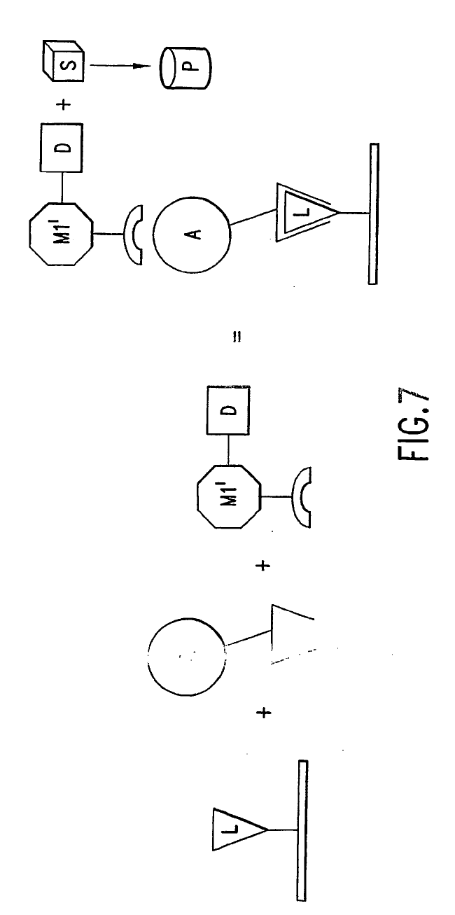

In another embodiment, a non-limiting example of which is shown schematically in FIG. 7, the present invention is a method for detecting or measuring an analyte that binds to a known ligand, comprising the steps of (a) contacting a first molecule that is known to be a ligand of a biomolecular analyte with a sample containing the analyte under conditions that allow the analyte to be bound by the first molecule; (b) removing unbound sample; (c) contacting the bound analyte with a second, different molecule that binds the analyte when the analyte is bound to the first molecule, under conditions that allow the analyte to be bound by the second molecule; (d) removing unbound second molecule; and (e) detecting or measuring binding of the second molecule to the analyte; wherein the first molecule is a ligand for the analyte and wherein the second molecules is a non-antibody protein that is derived from a species different from that of the analyte; wherein the first molecule is attached to a solid support either before or after step (a); and wherein detection or measurement of binding indicates presence or amount, respectively, of the analyte.

In a preferred embodiment, binding of the second molecule is detected or measured when the analyte is bound to the first molecule. In another preferred embodiment, binding is detected or measured when the analyte is bound, through the first molecule, to a solid support.

In another embodiment, the ligand and the binding molecule are contacted with the sample sequentially. In one embodiment, a step to remove unbound sample or binding molecule may be performed at any step prior to step (e).

In another embodiment, the analyte is an antibody. In yet another embodiment, the analyte is an antibody made by an individual with a disease and the production of said antibody is an indicator of the disease. In yet another embodiment, the analyte is an antibody made by a patient in response to an infectious organism. In yet another embodiment, the overproduction of said analyte is the cause of a disease. In yet another embodiment, the overproduction of said analyte is indicative of a disease. In yet another embodiment, one or more other different molecules may bind to the analyte of interest. Alternatively, in yet another embodiment, one or more other molecules can bind to the second molecule bound to the analyte. In yet other embodiments, secondary molecules that bind to the molecules that bind to the second molecule, or that bind to other secondary molecules, are also employed.

In another embodiment, the binding proteins used in a diagnostic assay are added simultaneously, omitting intermediate washing steps, but a final washing step is implemented to remove all unbound molecules prior to addition of certain detection molecules, such as substrate, in instances in which an enzymatic readout is used.

In yet another embodiment, all washing steps in a diagnostic test are omitted and the assay is formatted as a so-called “homogeneous assay” by any of several approaches well known in the art. Examples of homogeneous assays known in the art include, without limitation, (a) spin-labeled reporters, where binding of binding protein to the analyte is detected by a change in reporter mobility (broadening of the spin splitting peaks); (b) fluorescent reporters, where binding is detected by a change in fluorescence efficiency or by FRET (fluorescence energy transfer microscopy; e.g. Kenworthy, 2001, “Imaging protein-protein interactions using fluorescence resonance energy transfer microscopy”, Methods. 24:289-296); (c) scintillation proximity (e.g., Alderton and Lowe, 1999, “Scintillation proximity assay to measure nitroarginine and tetrahydrobiopterin binding to heme domain of neuronal nitric oxide synthase”, Methods Enzymol. 301:114-125); (d) enzyme reporters, where binding effects enzyme/substrate interactions; and (e) liposome-bound reporters, where binding leads to liposome lysis and release of encapsulated reporter (See, e.g., U.S. Pat. No. 6,214,970).

A molecule can be attached to a solid support using any technique known in the art for attaching a protein to a solid support. In one embodiment, the solid support is a polystyrene, 96-well microtiter plate. In another embodiment, the solid support is a composition that can be placed in a well (e.g., nickel-coated bead, antibody-conjugated Sepharose, magnetic particle). In yet another embodiment, the solid support is a well of a nanoarray device as described, for example, in PCT International Publication No. WO 0183827 to Yale University, published on Nov. 8, 2001, and in Zhu et al. (2000, “Analysis of yeast protein kinases using protein chips”, Nature Genet. 26:283-289).

A molecule that binds an analyte or that binds a molecule bound to the analyte can be from any source of proteins. In one embodiment, the molecule is derived from a collection of synthetic proteins. In another embodiment, the molecule is derived from a prokaryote. In another embodiment, the molecule is derived from a eukaryote. In another embodiment, the molecule is derived from a vertebrate. In another embodiment, the molecule is derived from a mammal. In another embodiment, the molecule is derived from a primate. In another embodiment, the molecule is derived from a rodent, insect, nematode or plant. In a specific embodiment, the molecule is derived from, for example, a monkey, fruit fly, cow, horse, sheep, pig, chicken, turkey, quail, cat, dog, mouse, rat or rabbit. In a specific embodiment, the molecule is derived from a yeast.

A molecule that binds an analyte or that binds another molecule bound to the analyte can be derived from the same or different species from that of the analyte or from that of said other molecules. However, use of a molecule derived from a species different from that of the analyte is advantageous for, inter alia, decreasing non-specific binding. For example, a molecule derived from a plant species may not have a homologous or orthologous gene product in the species from which the sample (containing the analyte of interest) is derived. Thus, use of such plant-derived molecules in binding assays to screen human-derived samples can produce more easily detectable signals and fewer false positives.

Accordingly, in one embodiment, at least one molecule that binds an analyte is derived from a species different from that of the analyte. In another embodiment, all different molecules that bind an analyte are derived from a species different from that of the analyte. In a specific embodiment, first and second molecules that bind an analyte are derived from a species different from that of the analyte.

In another embodiment, at least one molecule that binds an analyte is derived from a species different from that of another molecule that binds the analyte. In another embodiment, all different molecules that bind an analyte are derived from the same species, which species is different from that of the analyte. In yet another embodiment, all different molecules that bind an analyte are derived from species different from that of the analyte, and the molecules do not have homologous or orthologous gene products in the species from which the analyte is derived. In a specific embodiment, first and second molecules that bind an analyte are derived from the same species, which species is different from that of the analyte. In another specific embodiment, at least one of the first and second molecules that bind an analyte is derived from yeast. In another specific embodiment, all different molecules that bind an analyte are derived from yeast. In another specific embodiment, the analyte is human-derived, and the first molecule is derived from yeast. In yet another specific embodiment, the analyte is human-derived, and one of the first or second molecules is derived from yeast.

In certain instances, the analyte may not be specific to a particular species, such that use of a molecule that binds an analyte can be derived from any species. For example, some small molecules, inorganic compounds, carbohydrates, lipids, steroid hormones or non-naturally occurring compounds might not be derived from a species or might not exhibit species-specific expression. In such instances, the use of binding molecules from a species different from the one from which the sample to be tested for analyte was obtained can still provide an advantage since other molecules in the sample might be less likely to cross-react non-specifically with the binding molecules.

A molecule that binds an analyte or that binds a molecule bound to the analyte in the present invention can be an antibody or a non-antibody protein. Use of such non-antibody molecules in an assay to detect and/or measure an analyte has several advantages, however. Such non-antibody molecules can be used instead of antibodies in antibody-based diagnostic assays known in the art. Such non-antibody molecules can be particularly useful when the target analyte is weakly antigenic, since antibodies of sufficient specificity and affinity towards weakly antigenic analytes can be difficult to produce.

Accordingly, in one embodiment, two, three or four of the molecules that bind an analyte or a molecule bound to the analyte are non-antibody proteins. In another embodiment, all different molecules that bind an analyte or a molecule bound to the analyte are non-antibody proteins. In another embodiment, a first molecule that binds an analyte is a non-antibody protein and a second different molecule that binds the first molecule when bound to the analyte is an antibody, preferably a monoclonal antibody. In another embodiment, a first molecule that binds an analyte and a second different molecule that binds the first molecule when bound to the analyte are non-antibody proteins. In a further embodiment, the first and second molecules are non-antibody proteins derived from a species different from that of the analyte. In a non-limiting embodiment, an antibody that binds to a non-antibody binding protein serves as a reporter molecule by virtue of its conjugation to a detectable molecule.

A molecule that binds an analyte or a molecule bound to the analyte can be a chimeric protein, fusion protein, full-length protein, portion of a protein or peptide. In one embodiment, one, two, three or four different molecules are used in an assay to detect and/or measure an analyte.

A molecule that binds an analyte or a molecule bound to the analyte can have a detectable marker conjugated to it, or can be bound by a detectable marker. In one embodiment, such a molecule is conjugated to a detectable marker. In another embodiment, such a molecule is conjugated to an enzyme (e.g., horseradish peroxidase, alkaline phosphatase, luciferase) that produces a detectable marker. In another embodiment, such a molecule is conjugated to a hapten (e.g., biotin, avidin). In another embodiment, such a molecule is bound to a detectable marker.

Molecules useful for the assays of the present invention include such molecules identified by screening protein arrays. Examples of arrays useful for identifying and isolating molecules that bind an analyte or a molecule bound to the analyte, and binding molecules identified using such arrays, are described in PCT International Publication No. WO 0183827 to Yale University, published on Nov. 8, 2001, which is incorporated herein by reference in its entirety. See also Zhu et al. (2001, “Global analysis of protein activities using proteome chips”, Science. 293:2101-2105), Zhu and Snyder (2001, “Protein arrays and microarrays”, Curr Opin Chem Biol. 5:40-45) and Zhu et al. (2000, “Analysis of yeast protein kinases using protein chips”, Nature Genet. 26:283-289) and include, but are not limited to, proteins, nucleic acids and lipids.

Accordingly, in one embodiment, a protein that binds an analyte (or that binds another molecule when bound to the analyte, or that binds a second molecule when bound to a different first molecule) is identified by screening a protein array comprising at least one protein encoded by at least 50% or at least 70% of the known genes in a single species is used in an assay of the present invention. In another embodiment, such a protein is identified by screening a protein array comprising at least 50% of all proteins expressed in a single species is used (such that protein isoforms and splice variants are counted as a single protein). In another embodiment, such a protein is identified by screening a protein array comprising at least 1000 proteins expressed in a single species is used. In yet another embodiment, such a protein is identified by screening a protein array comprising proteins encoded by at least 1000 different known genes in a single species is used.

Samples, potentially containing analyte that are useful for the assays of the present invention include, but are not limited to, an aqueous solution, soil, food, fecal matter, plant or animal cells, tissue or tissue extract, tissue culture, tissue culture extract or tissue culture medium. In one embodiment, the sample to be assayed for the presence and/or amount of an analyte is a patient sample. In a further embodiment, the patient sample is a biological fluid such as, but not limited to, blood, serum, lymph, plasma, milk, urine, saliva, pleural effusions, synovial fluid, spinal fluid, tissue infiltrations or tumor infiltrates. In another embodiment, the patient sample is a tissue or tissue extract. In yet another embodiment, the patient sample is fecal matter. In a specific embodiment, the sample tissue is obtained from a biopsy.

An analyte preferably is a biomolecule. An analyte can also be, without limitation, an intact cell or a component of the cell. However, an analyte can also be a small molecule (e.g., steroid, pharmaceutical drug). A small molecule is considered a non-peptide compound with a molecular weight of less than 500 daltons. Although the analyte in a preferred embodiment of the present invention is an organic molecule, and more preferably a biomolecule, analytes in other embodiments of this invention are non-biomolecules, including, but not limited to, minerals, toxic inorganic compounds, inorganic pollutants, non-biological allergens and the like. In one specific embodiment of the present invention, the analyte is lead. In another specific embodiment, the analyte is lead and the sample to be tested for the presence of lead is obtained from a human patient.

Thus, for example, a small molecule can be a human-derived steroid hormone such as, but not limited to, adrenalin, noradrenalin, glucocorticoid, mineralocorticoid, cortical sex hormone, androgen (e.g., testosterone), estrogen (e.g., estradiol) or progestin (e.g., progesterone).

A diagnostic assay could be designed to detect a hormone analyte using binding proteins obtained from a collection of plant proteins. Binding proteins from a plant protein array would likely be derived from a species different from that in which the above-identified human-derived steroid hormones are derived, for example, as most mammalian hormones are not present in plants. For example, a steroid hormone can be used to screen a protein array comprising a group of proteins derived from the plant, Arabadopsis thaliana. Any binding proteins identified by this screening method can be used as binding proteins in a diagnostic assay for detection and identification of such hormone analytes.

Examples of analytes include, but are not limited to, bacteria, viruses, antigens, antibodies, and polynucleotides. Particularly useful analytes are, for example, proteins, carbohydrates and lipids whose presence or levels correlate with a disease or disorder. The presence or levels of such analytes may correlate with the risk, onset, progression, amelioration and/or remission of a disease or disorder.

Accordingly, the analyte can be a protein, peptide, amino acid, nucleic acid, carbohydrate or lipid, including a fatty acid. In one embodiment, the analyte is a polypeptide having a modification such as, but not limited to, phosphorylation, glycosylation or acylation. In another embodiment, the analyte is a synthetic peptide, oligonucleotide or fatty acid.

In a particular embodiment, the analyte is a human-derived hormone such as, but not limited to, gastrin, secretin, cholecystokinin, insulin, glucagon, thyroxine triiodothyronine, calcitonin, parathyroid hormone, thymo sin, releasing hormones, oxytocin, vasopressin, growth hormone, prolactin, melanophore-stimulating hormone, thyrotrophic hormone, adrenocorticotrophic hormone, follicle-stimulating hormone, luteinizing hormone, or melatonin.

In one embodiment, the analyte is a marker for a disease or disorder. Such disease or disorder can be, without limitation, an allergy, anxiety disorder, autoimmune disease, behavioral disorder, birth defect, blood disorder, bone disease, cancer, circulatory disease, tooth disease, depressive disorder, dissociative disorder, ear condition, eating disorder, eye condition, food allergy, food-borne illness, gastrointestinal disease, genetic disorder, heart disease, hormonal disorder, immune deficiency, infectious disease, inflammatory disease or disorder, insect-transmitted disease, nutritional disorder, kidney disease, leukodystrophy, liver disease, mental health disorder, metabolic disease, mood disorder, musculodegenerative disorder, neurological disorder, neurodegenerative disorder, neuromuscular disorder, personality disorder, phobia, pregnancy complication, prion disease, prostate disease, psychological disorder, psychiatric disorder, respiratory disease, sexual disorder, skin condition, sleep disorder, speech-language disorder, sports injury, tropical disease, vestibular disorder or wasting disease.

In another embodiment, the analyte is a marker for an infection or infectious disease such as, but not limited to, acquired immunodeficiency syndrome (AIDS/HIV) or HIV-related disorders, Alpers syndrome, anthrax, bovine spongiform encephalopathy, (BSE), chicken pox, cholera, conjunctivitis, Creutzfeldt-Jakob disease (CJD), dengue fever, ebola, elephantiasis, encephalitis, fatal familial insomnia, Fifth's disease, Gerstmann-Straussler-Scheinker syndrome, hantavirus, helicobacter pylori, hepatitis (hepatitis A, hepatitis B, hepatitis C), herpes, influenza, Kuru, leprosy, lyme disease, malaria, hemorrhagic fever (e.g., Rift Valley fever, Crimean-Congo hemorrhagic fever, Lassa fever, Marburg virus disease, and Ebola hemorrhagic fever), measles, meningitis (viral, bacterial), mononucleosis, nosocomial infections, otitis media, pelvic inflammatory disease (PID), plague, pneumonia, polio, prion disease, rabies, rheumatic fever, roseola, Ross River virus infection, rubella, salmonellosis, septic arthritis, sexually transmitted diseases (STDs), shingles, smallpox, strep throat, tetanus, toxic shock syndrome, toxoplasmosis, trachoma, tuberculosis, tularemia, typhoid fever, valley fever, whooping cough or yellow fever.

In another embodiment, the analyte is a marker for an autoimmune disease such as, but not limited to, Addison's disease, alopecia areata, ankylosing spondylitis, antiphospholipid syndrome (APS), Behcet's disease, chronic fatigue syndrome, Crohn's disease and ulcerative colitis, fibromyalgia, Goodpasture syndrome, graft versus host disease, Lupus (e.g., Systemic lupus erythematosus), Meniere's disease, multiple sclerosis, myasthenia gravis, myositis, pemphigus vulgaris, psoriasis, rheumatic fever, sarcoidosis, scleroderma, vasculitis, vitiligo or Wegener's granulomatosis.

In another embodiment, the analyte is a marker for a birth defect such as, but not limited to, Aicardi syndrome, albinism, anencephaly, CHARGE syndrome, cleft palate, fetal alcohol syndrome (FAS), hypospadias, spina bifida, thrombocytopenia absent radius (TAR) syndrome or trisomy.

In another embodiment, the analyte is a marker for a blood disorder such as, but not limited to, anemia, antiphospholipid syndrome (APS), blue rubber bleb nevus syndrome,
gout, hemophilia, leukemia, myeloproliferative disorders, sepsis, sickle cell disease or thalassemia.

In another embodiment, the analyte is a marker for a bone disease such as, but not limited to, achondroplasia, bone cancer, fibrodysplasia ossificans progressiva, fibrous dysplasia, Legg-Calve-Perthes disease, myeloma, osteoarthritis, osteogenesis imperfecta, osteoporosis, Paget's disease or scoliosis.

In another embodiment, the analyte is a marker for a circulatory disease such as, but not limited to, elephantiasis, heart disease, hemochromatosis, hemophilia, hypertension, hypotension, Klippel-Trenaunay-Weber syndrome, lymphedema, neutropenia, peripheral vascular disease (PVD), phlebitis, Raynaud's phenomenon, thrombosis, twin-to-twin transfusion syndrome or vasculitis.

In another embodiment, the analyte is a marker for a metabolic disease such as, but not limited to, acid maltase deficiency, diabetes, galactosemia, hypoglycenia, Lesch-Nyhan syndrome, maple syrup urine disease (MSUD), Niemann-Pick disease, phenylketonuria or urea cycle disorder.

In another embodiment, the analyte is a marker for a nutrition or gastrointestinal disorder such as, but not limited to, appendicitis, botulism, canker sores, celiac disease, colitis (including ulcerative colitis), cyclic vomiting syndrome (CVS), diarrhea, hiatus hernia, inflammatory bowel disease (IBD), irritable bowel syndrome (IBS), peptic ulcer, primary biliary cirrhosis, salinonellosis, anorexia nervosa, bulimia nervosa, bovine spongiform encephalopathy (BSE), Fugu poisoning or diverticulitis.

In another embodiment, the analyte is a marker for an ear disorder such as, but not limited to, acoustic neuroma, cholesteatoma, deafness, mastoiditis, Meniere's disease, otitis, tinnitus or a vestibular disorder.

In another embodiment, the analyte is a marker for an eye disorder such as, but not limited to, amblyopia, cataract, color blindness, conjunctivitis, glaucoma, keratoconus, macular degeneration, microphthalmia, anophthalmia, retinitis pigmentosa, retinoblastoma; strabismus or trachoma.

In another embodiment, the analyte can be a marker for a genetic disorder such as, but not limited to, achondroplasia, achromatopsia, acid maltase deficiency, adrerioleukodystrophy, Aicardi syndrome, alpha-1 antitrypsin deficiency, androgen insensitivity syndrome, Apert syndrome, arrhythmogenic right ventricular dysplasia, ataxia relangiectasia, Canavan disease, Cri Du Chat syndrom, cystic fibrosis, Dercum's disease, familial adenomatous polyposis, familial breast cancer susceptibility, Fanconi anemia, fragile X syndrome, galactosemia, Gaucher disease, hemochromatosis, Huntington's disease, Hurler syndrome, hypophosphatasia, Klinefelter syndrome, Krabbes disease, Langer-Giedion syndrome, leukodystrophy, long QT syndrome, Marfan syndrome, Moebius syndrome, mucopolysaccharidosis (MPS), nail patella syndrome, nephrogenic diabetes insipidus, porphyria, non-hereditary polyposis colorectal cancer (NHPCC), Prader-Willi syndrome, progeria, Proteus syndrome, Rett syndrome, Rubinstein-Taybi syndrome, Sanfilippo syndrome, Shwachman syndrome, Smith-Magenis syndrome, Stickler syndrome, Tay-Sachs disease, Treacher Collins syndrome, triose phosphate isomerase deficiency, trisomy, tuberous sclerosis, Turner's syndrome, urea cycle disorder, Williams syndrome, Wilson's disease or angina pectoris.

In another embodiment, the analyte can be a marker for a heart disease such as, but not limited to, arrhythmogenic right ventricular dysplasia, atherosclerosis/arteriosclerosis, cardiomyopathy, congenital heart disease, endocarditis, enlarged heart, heart attack, heart failure, heart murmur, heart palpitations, high cholesterol, high tryglycerides, hypertension, long QT syndrome, mitral valve prolapse, postural orthostatic tachycardia syndrome, tetralogy of fallots or thrombosis.

In another embodiment, the analyte can be a marker for a kidney disorder such as, but not limited to, kidney cancer, kidney infection, kidney stones, kidney transplants, nephrogenic diabetes insipidus, nephrology or rhabdomyolysis.

In another embodiment, the analyte can be a marker for a leukodystrophy such as, but not limited to, adrenoleukodystrophy and Krabbes disease.

In another embodiment, the analyte can be a marker for a liver disorder such as, but not limited to, alpha-1 antitrypsin deficiency, Gilbert's syndrome, hepatitis or liver cancer.

In another embodiment, the analyte can be a marker for a mood disorder such as, but not limited to, bipolar disorder (manic depression), depressive disorder or seasonal affective disorder.

In another embodiment, the analyte can be a marker for a neurological or musculoskeletal disorder such as, but not limited to, Aicardi syndrome, Alzheimer's disease, amnesia, amyotrophic lateral sclerosis (Lou Gehrig's Disease), anencephaly, aphasia, arachnoiditis, Arnold Chiari malformation, ataxia telangiectasia, Batten disease, Bell's palsy, brachial plexus injury, brain injury, brain tumor, Charcol-Marie-Tooth disease, encephalitis, epilepsy, essential tremor, Guillain-Barre Syndrome, hydrocephalus, hyperhidrosis, Krabbes disease, meningitis, Moebius syndrome, muscular dystrophy, multiple sclerosis, Parkinson's disease, peripheral neuropathy, postural or orthostatic tachycardia syndrome, progressive supranuclear palsy, Reye's syndrome, shingles, Shy-Drager Syndrome (SDS), spasmodic torticollis, spina bifida, spinal muscular atrophy, Stiff Man syndrome, synesthesia, syringomyelia, thoracic outlet syndrome, Tourette syndrome, toxoplasmosis or trigeminal neuralgia

In another embodiment, the analyte can be a marker for a respiratory disease such as, but not limited to, alveolar capillary dysplasia, asthma, black lung, bronchiolitis, chronic obstructive pulmonary disease (COPD), emphysema, laryngeal cancer, laryngomalacia, legionnaires' disease, lung cancer, lymphagioleiomyomatosis (LAM), pleurisy (pleuritis), pneumonia, respiratory distress syndrome, respiratory syncytial virus (RSV), sarcoidosis, silicosis, sinus infection, tonsillitis, tuberculosis or valley fever.

In another embodiment, the analyte can be a marker for a skin condition such as, but not limited to, chicken pox, chronic hives (urticaria), decubitus ulcer, eczema, Ehlers-Danlos Syndrome, epidermolysis bullosa, gangrene, hidradenitis suppurativa, hot tub folliculitis, hyperhidrosis, ichthyosis, impetigo, keratosis pilaris, leprosy, measles, molluscum contagiosum, pityriasis rosea, porphyria, pseudofolliculitis barbae, psoriasis, rosacea, rubella, scleroderma, shingles or skin cancer.

In another embodiment, the analyte can be a marker for a tropical disease such as, but not limited to, Chagas disease, cholera, dengue fever, diphtheria, dysentery (bacterial or ameboe), ebola, encephalitis, giardiasis, Lassa fever, leishmaniasis, leprosy, malaria, Marburg hemorrhagic fever, meningitis, polio, Ross River virus infection, schistosomiasis, tetanus, tuberculosis, typhoid fever, typus or yellow fever.

An analyte can be a component of a virus such as, but not limited to, herpes simplex virus, cytomegalovirus, Epstein-Barr virus, human immunodeficiency virus-1, adenovirus, rhinovirus, human immunodeficiency virus-2, human papilloma virus, HTLV-I, HTLV-II or HTLV-III. Accordingly, in another embodiment, the analyte can be a component of a virus, wherein the virus is a member of a family such as, but not limited to, the Poxviridae, Iridoviridae, Herpesviridae, Adenoviridae, Papovaviridae, Hepadnaviridae, Parvoviridae, Reoviridae, Birnaviridae, Togaviridae, Coronaviridae, Paramyxoviridae, Rhabdoviridae, Filoviridae, Orthomyxoviridae, Bunyaviridae, Arenaviridae, Retroviridae, Picornaviridae, Calciviridae or Chlamydia.

In another embodiment, the analyte is a marker for cancer such as, but not limited to, non-Hodgkin's lymphoma, Hodgkin's lymphoma, leukemia (e.g., acute lymphocytic leukemia, acute myelocytic leukemia, chronic myeloid leukemia, chronic lymphocytic leukemia, multiple myeloma), colon carcinoma, rectal carcinoma, pancreatic cancer, breast cancer, ovarian cancer, prostate cancer, renal cell carcinoma, hepatic cancer, bile duct carcinoma, choriocarcinoma, cervical cancer, testicular cancer, lung carcinoma, bladder carcinoma, melanoma, head and neck cancer, brain cancer, cancers of unknown primary site, neoplasms, cancers of the peripheral nervous system, cancers of the central nervous system; and other tumor types and subtypes (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, seminoma, embryonal carcinoma, Wilms' tumor, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, and retinoblastoma), heavy chain disease, metastases, or any disease or disorder characterized by uncontrolled or abnormal cell growth.

In a specific embodiment, the analyte is the ras protein.

In another embodiment, the analyte is a marker for a protozoal disease such as those caused by, without limitation, Kinetoplastida such as Trypanosoma and Leishmania, Diplomonadina such as Giardia, Trichomonadida such as Dientamoeba and Trichomonas, Gymnamoebia such as Naegleria and the Amoebida such as Entamoeba and Acanthamoeba, Sporozoasida such as Babesia and the Coccidiasina such as Isospora, Toxoplasma, Cryptosporidium, Eimeria, Thelleria, and Plasmodium.

In another embodiment, the analyte is a marker for a metazoal disease such as those caused by, without limitation, the Nematoda (roundworms) such as Ascaris, Toxocara, the hookworms, Strongyloides, the whipworms, the pinworms, Dracunculus, Trichinella, and the filarial worms, and by the Platyhelminthes (flatworms) such as the Trematoda such as Schistosoma, the blood flukes, liver flukes, intestinal flukes, and lung flukes, and the Cestoda such as the tapeworms.

In another embodiment, the analyte is a marker for a bacterial disease (e.g., caused by group B streptococci, Listeria monocytogenes, Neisseria meningitidis, staphylococci, salmonella, or Escherichia coli), mycobacterial disease, spirochetal disease, chlamydia, rickettsial disease or fungal disease.

Also, analyte marker for other conditions can be assayed such as, but not limited to, pregnancy, alcoholism, drug abuse, allergy, poisoning, secondary effects of, or responses to, treatments or secondary effects of diseases.

A detectable marker can be visible to the naked eye or visualized with the aid of an optical filter. As such, a detectable marker can be a colorimetric label including, without limitation, metallic sol particles, gold sol particles, dye sol particles, dyed latex particles and dyes encapsulated in liposomes. Other detectable markers include, but are not limited to, radionuclides, fluorescent moieties, and luminescent moieties. In one embodiment, a molecule that binds an analyte of interest is conjugated to a detectable marker. In another embodiment, a molecule conjugated to a detectable marker binds a molecule that binds an analyte either directly or indirectly.

In another embodiment, a molecule that binds an antalyte, or that binds another molecule that binds an analyte, is conjugated to an enzyme that can be detected or can produce a detectable marker. In a preferred embodiment, a molecule that binds another molecule that binds to the analyte is conjugated to such an enzyme. Many enzymes known in the art can be useful for the assays of the invention (see, e.g., Engvall, 1980, “Enzyme Immunoassay ELISA and EMIT”, Methods of Enzymology, 70:419-439). Accordingly, a molecule that binds the analyte or that binds another molecule that binds to the analyte can be conjugated to, for example, alkaline phosphatase, glucose oxidase, beta-galactosidase, horseradish peroxidase, lysozynie, glucose-6-phosphate dehydrogenase, lactate dehydrogenase or urease.

In another embodiment, a detectable marker is contacted with the complex formed by binding of a molecule and an analyte, and then directly visualized. For example, a fluorescently tagged antibody directed to a molecule that binds the analyte, or directed to the complex, can be bound to the complex, and then be detected by epifluorescence.

Binding of an analyte to a molecule can be detected by, for example, using radioactively-labeled ligand followed by autoradiography and/or phosphoimager analysis; binding of hapten, which is then detected by a fluorescently labeled or enzymatically labeled antibody or high-affinity hapten ligand such as biotin or streptavidin; mass spectrometry; atomic force microscopy; fluorescent polarization methods; infrared-labeled compounds or proteins; radioactively-labeled, fluorescently labeled or amplifiable oligonucleotides; stimulation or inhibition of biological activity of an analyte or a molecule that binds either the analyte or another molecule that binds the analyte; rolling circle amplification-detection methods; competitive PCR; calorimetric procedures; or biological assays (e.g., for virus titers).

In one embodiment, the value obtained by an assay of the invention is quantitative. In another embodiment, the value obtained by the assay is semi-quantitative or qualitative (i.e., above or below a threshold value).

The binding assays can be carried out in various formats including, for example, a 96-well format (e.g., microtiter plate), which is preferred for carrying out the assays in a batch mode. Also preferred for batch mode analysis is a nanoarray device such as, without limitation, the nanoarray device described in PCT International Publication No. WO 0183827, published on Nov. 8, 2001, and in Zhu et al. (2000, “Analysis of yeast protein kinases using protein chips”, Nature Genet. 26:283-289). The assays can be carried out in an automated assay analyzer (e.g., of the continuous/random access type) which can perform assays on many different samples, and are well known in the art. Examples of such automated assay analyzers are described in U.S. Pat. Nos. 5,207,987 and 5,518,688 to PB Diagnostic Systems, Inc. Automated assay analyzers that are commercially available include, for example, the OPUS.R™, OPUS MAGNUM.R™, Vitros™ (Ortho), Elecsys™ (Roche), AxSYM™ (Abbott), Prism™ (Abbott), Architect™ (Abbott), Centaur™ (Bayer) and Immuno 1™ (Bayer).

Another assay format that can be used in accordance with the present invention is a rapid manual test, which can be administered at the location (e.g., doctor's office) where the sample is obtained.

One or more of the molecules (i.e., a molecule that binds the analyte or that binds another molecule that binds to the analyte) and/or analytes can be bound to a solid support. The solid support can comprise glass, ceramics, amorphous silicon carbide, castable oxides, polyimides, polymethylmethacrylates, polystyrenes, silicone elastomers, nitrocellulose, acrylamide, agarose or gold. In one embodiment, the solid support comprises wells. In a specific embodiment, the solid support is a 96-well microtiter plate. In another specific embodiment, the solid support is a nanoarray device.

In another embodiment, the solid support can be contained within a well. In a particular embodiment, the solid support can be a magnetic particle. In another particular embodiment, the solid support can be a polystyrene bead. An analyte and/or a molecule that binds the analyte or that binds another molecule that binds to the analyte can be bound directly to the solid support, or can be attached to the solid support through a linker compound. The linker can be any compound that derivatizes the surface of the solid support to facilitate the attachment to the surface of the solid support of an analyte or a molecule that binds the analyte or that binds another molecule that binds to the analyte. The linker may covalently or non-covalently bind one such molecule and/or the analyte to the surface of the solid support. In addition, the linker can be an inorganic or organic compound.

In another embodiment, the solid support comprises a material that helps bind the binding molecules and/or analytes to the solid support. For example, the solid support can be coated with a material that binds to an affinity tag on a molecule that binds an analyte of interest. In a specific embodiment, the solid support comprises glutathione. In another specific embodiment, the solid support comprises nickel. In another specific embodiment, the solid support comprises glutathione and nickel.

5.2. Method for Determining a Diagnosis or Prognosis of a Disease or Disorder by Detecting an Analyte Using a Non-Antibody Molecule that Binds the Analyte

The invention also relates to a method for diagnosing a disease or disorder in a subject. Accordingly, in one embodiment, the present invention provides for a method of diagnosing a disease or disorder in a subject comprising the steps of (a) contacting a first molecule that binds a biomolecular analyte with a sample that might or might not contain an analyte, from the subject under conditions that allow the analyte to be bound by the first molecule; (b) contacting any bound analyte present with a second, different molecule that binds the analyte when the analyte is bound to the first molecule, under conditions that allow the analyte to be bound by the second molecule; (c) detecting or measuring binding of the second molecule to the analyte, wherein detection or measurement of binding indicates presence or amount, respectively, of the analyte; wherein at least one of the first and second molecules is a non-antibody protein that is derived from a species different from that of the analyte; wherein the first molecule is attached to a solid support either before or after step (a); and wherein the disease or disorder is determined to be present when the presence, absence or amount of analyte in step (c) differs from a control value representing the amount of analyte present in an analogous sample from a subject not having the disease or disorder. In a further embodiment, prior to step (a), the method comprises the step of attaching the first molecule to a solid support.

In a preferred embodiment, the first and second molecules are non-antibody proteins that are derived from a species different from that of the analyte. In another preferred embodiment, binding of the second molecule is detected or measured when the analyte is bound to the first molecule. In another preferred embodiment, binding of the second molecule is detected or measured when the analyte is bound, through the first molecule, to a solid support.

In a further embodiment, the unbound sample is removed prior to step (c). In another further embodiment, the unbound first or second molecule is removed prior to step (e). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules (e.g., contaminants, excess reagents) that are not present in a complex comprising analyte, first molecule and second molecule.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, the present invention provides for a method of diagnosing a disease or disorder in a subject comprising the steps of (a) contacting a first molecule that binds a biomolecular analyte with a sample that might or might not contain an analyte, from the subject under conditions that allow the analyte to be bound by the first molecule; (b) removing unbound sample; (c) contacting any bound analyte present with a second, different molecule that binds the analyte when the analyte is bound to the first molecule, under conditions that allow the analyte to be bound by the second molecule; (d) removing unbound second molecule; and (e) detecting or measuring binding of the second molecule to the analyte, wherein detection or measurement of binding indicates presence or amount, respectively, of the analyte; wherein at least one of the first and second molecules is a non-antibody protein that is derived from a species different from that of the analyte; wherein the first molecule is attached to a solid support either before or after step (a); and wherein the disease or disorder is determined to be present when the presence, absence or amount of analyte in step (e) differs from a control value representing the amount of analyte present in an analogous sample from a subject not having the disease or disorder. In a further embodiment, prior to step (a), the method comprises the step of attaching the first molecule to a solid support.

In a preferred embodiment, the first and second molecules are non-antibody proteins that are derived from a species different from that of the analyte. In another preferred embodiment, binding of the second molecule is detected or measured when the analyte is bound to the first molecule. In another preferred embodiment, binding of the second molecule is detected or measured when the analyte is bound, through the first molecule, to a solid support.

In an alternate embodiment, a step to remove unbound sample or binding molecule may be performed at any step prior to step (e). For example, several steps prior to step (e) can be introduced, in lieu of steps (b) and (d), to selectively remove unbound sample, unbound first molecules or unbound second molecules.

In another embodiment, the present invention provides for a method of diagnosing a disease or disorder in a subject comprising the steps of (a) contacting a first molecule that binds a biomolecular analyte with a sample, that might or might not contain an analyte, from the subject under conditions that allow the analyte to be bound by the first molecule; (b) contacting the bound, first molecule with a second, different molecule that binds the first molecule when the first molecule is bound to the analyte, under conditions that allow the first molecule to be bound by the second molecule; and (c) detecting or measuring binding of the second molecule to the first molecule, wherein detection or measurement of binding indicates presence or amount, respectively, of the analyte; wherein the first molecule is a non-antibody protein that is derived from a species different from that of the analyte; wherein the first molecule is attached to a solid support either before or after step (a); and wherein the disease or disorder is determined to be present when the absence, presence or amount of analyte in step (c) differs from a control value representing the amount of analyte present in an analogous sample from a subject not having the disease or disorder. In a further embodiment, prior to step (a), the method comprises the step of attaching a first molecule to a solid support.

In a further embodiment, the unbound sample is removed prior to step (c). In another further embodiment, the unbound first or second molecule is removed prior to step (c). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules (e.g., contaminants, excess reagents) that are not present in a complex comprising analyte, first molecule and second molecule.

In a preferred embodiment, binding of the second molecule is detected or measured when the analyte is bound to the first molecule. In another preferred embodiment, binding of the second molecule is detected or measured when the analyte is bound, through the first molecule, to a solid support.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, the present invention provides for a method of diagnosing a disease or disorder in a subject comprising the steps of (a) contacting a first molecule that binds a biomolecular analyte with a sample, that might or might not contain an analyte, from the subject under conditions that allow the analyte to be bound by the first molecule; (b) removing unbound sample; (c) contacting the bound, first molecule with a second, different molecule that binds the first molecule when the first molecule is bound to the analyte, under conditions that allow the first molecule to be bound by the second molecule; (d) removing unbound second molecule; and (e) detecting or measuring binding of the second molecule to the first molecule, wherein detection or measurement of binding indicates presence or amount, respectively, of the analyte; wherein the first molecule is a non-antibody protein that is derived from a species different from that of the analyte; wherein the first molecule is attached to a solid support either before or after step (a); and wherein the disease or disorder is determined to be present when the absence, presence or amount of analyte in step (e) differs from a control value representing the amount of analyte present in an analogous sample from a subject not having the disease or disorder. In a further embodiment, prior to step (a), the method comprises the step of attaching a first molecule to a solid support.

In a preferred embodiment, binding of the second molecule is detected or measured when the analyte is bound to the first molecule. In another preferred embodiment, binding of the second molecule is detected or measured when the analyte is bound, through the first molecule, to a solid support.

In an alternate embodiment, a step to remove unbound sample or binding molecule may be performed at any step prior to step (e). For example, several steps prior to step (e) can be introduced, in lieu of steps (b) and (d), to selectively remove unbound sample, unbound first molecules or unbound second molecules.

The present invention also encompasses methods for determining a prognosis for a disease, disorder or other condition. Also, prognostic markers for the response (toxic or ameliorative) can be assayed to provide information important for treatment course and dosages. Prognosis of a disease or determination of possible response to a therapeutic treatment generally involves staging of the disease or disorder. For example, a baseline can be determined prior to manifestation of any symptoms, at a point in the progression of the disease or disorder, or before, during or after therapeutic intervention.

Accordingly, in one embodiment, the present invention provides a method for staging a disease or disorder in a subject comprising the steps of (a) contacting a first molecule that binds a biomolecular analyte with a sample, that might or might not contain an analyte, from the subject under conditions that allow the analyte to be bound by the first molecule, (b) contacting the bound analyte with a second, different molecule that binds the analyte when the analyte is bound to the first molecule, under conditions that allow the analyte to be bound by the second molecule, and (c) detecting or measuring binding of the second molecule to the analyte, wherein detection or measurement of binding indicates absence, presence or amount, respectively, of the analyte, wherein at least one of the first and second molecules is a non-antibody protein that is derived from a species different from that of the analyte; wherein the first molecule is attached to a solid support either before or after step (a); and wherein the stage of a disease or disorder in a subject is determined when the presence or amount of analyte in step (c) is compared with the amount of analyte present in an analogous sample from a subject having no disease and/or disorder or having a particular stage of the disease or disorder. In a further embodiment, prior to step (a), the method comprises the step of attaching a first molecule to a solid support.

In a further embodiment, the unbound sample is removed prior to step (c). In another further embodiment, the unbound first or second molecule is removed prior to step (c). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules (e.g., contaminants, excess reagents) that are not present in a complex comprising analyte, first molecule and second molecule.

In a preferred embodiment, the first and second molecules are non-antibody proteins that are derived from a species different from that of the analyte. In another preferred embodiment, binding of the second molecule is detected or measured when the analyte is bound to the first molecule. In another preferred embodiment, binding of the second molecule is detected or measured when the analyte is bound, through the first molecule, to a solid support.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, the present invention provides a method for staging a disease or [symbol] subject comprising the steps of (a) contacting a [symbol] molecule that binds a biomolecular analyte with a sample, that might or might not contain an analyte, from the subject under conditions that allow the analyte to be bound by the first molecule, (b) removing unbound sample, (c) contacting the bound analyte with a second, different molecule that binds the analyte when the analyte is bound to the first molecule, under conditions that allow the analyte to be bound by the second molecule, (d) removing unbound second molecule, and (e) detecting or measuring binding of the second molecule to the analyte, wherein detection or measurement of binding indicates absence, presence or amount, respectively, of the analyte, wherein at least one of the first and second molecules is a non-antibody protein that is derived from a species different from that of the analyte; wherein the first molecule is attached to a solid support either before or after step (a); and wherein the stage of a disease or disorder in a subject is determined when the presence or amount of analyte in step (e) is compared with the amount of analyte present in an analogous sample from a subject having no disease and/or disorder or having a particular stage of the disease or disorder. In a further embodiment, prior to step (a), the method comprises the step of attaching a first molecule to a solid support.

In a preferred embodiment, the first and second molecules are non-antibody proteins that are derived from a species different from that of the analyte. In another preferred embodiment, binding of the second molecule is detected or measured when the analyte is bound to the first molecule. In another preferred embodiment, binding of the second molecule is detected or measured when the analyte is bound, through the first molecule, to a solid support.

In an alternate embodiment, a step to remove unbound sample or binding molecule may be performed at any step prior to step (e). For example, several steps prior to step (e) can be introduced, in lieu of steps (b) and (d), to selectively remove unbound sample, unbound first molecules or unbound second molecules.

In another embodiment, the present invention provides a method for staging a disease or disorder in a subject comprising the steps of (a) contacting a first molecule that binds a biomolecular analyte with a sample, that might or might not contain an analyte, from the subject under conditions that allow the analyte to be bound by the first molecule; (b) contacting the bound, first molecule with a second, different molecule that binds the first molecule when the first molecule is bound to the analyte, under conditions that allow the first molecule to be bound by the second molecule; and (c) detecting or measuring binding of the second molecule to the first molecule, wherein detection or measurement of binding indicates presence or amount, respectively, of the analyte; wherein the first molecule is a non-antibody protein that is derived from a species different from that of the analyte; wherein the first molecule is attached to a solid support either before or after step (a); and wherein the stage of a disease or disorder in a subject is determined when the absence, presence or amount of analyte in step (c) is compared with the amount of analyte present in an analogous sample from a subject having no disease and/or disorder or having a particular stage of the disease or disorder. In a further embodiment, prior to step (a), the method comprises the step of attaching a first molecule to a solid support.

In a further embodiment, the unbound sample is removed prior to step (c). In another further embodiment, the unbound first or second molecule is removed prior to step (c). In another further embodiment, the unbound first and second molecules are removed prior to step (c). In another further embodiment, the unbound sample and the unbound first and second molecules are removed prior to step (c). In yet another further embodiment, one or more steps are added prior to step (c) to remove molecules (e.g., contaminants, excess reagents) that are not present in a complex comprising analyte, first molecule and second molecule.

In a preferred embodiment, binding of the second molecule is detected or measured when the analyte is bound to the first molecule. In another preferred embodiment, binding of the second molecule is detected or measured when the analyte is bound, through the first molecule, to a solid support.

In an alternate embodiment, a step to remove unbound sample or binding molecule may be performed at any step prior to step (e). For example, several steps prior to step (e) can be introduced, in lieu of steps (b) and (d), to selectively remove unbound sample, unbound first molecules or unbound second molecules.

The binding molecules can be contacted with the sample simultaneously. Alternatively, the binding molecules can be contacted with the sample sequentially. The binding molecules can be contacted with the sample in any sequence. Also, different binding molecules can be contacted with each other, in any sequence, prior to contacting the binding molecules with the sample suspected of containing an analyte of interest.

In another embodiment, the present invention provides a method for staging a disease or disorder in a subject comprising the steps of (a) contacting a first molecule that binds a biomolecular analyte with a sample, that might or might not contain an analyte, from the subject under conditions that allow the analyte to be bound by the first molecule; (b) removing unbound sample; (c) contacting the bound, first molecule with a second, different molecule that binds the first molecule when the first molecule is bound to the analyte, under conditions that allow the first molecule to be bound by the second molecule; (d) removing unbound second molecule; and (e) detecting or measuring binding of the second molecule to the first molecule, wherein detection or measurement of binding indicates presence or amount, respectively, of the analyte; wherein the first molecule is a non-antibody protein that is derived from a species different from that of the analyte; wherein the first molecule is attached to a solid support either before or after step (a); and wherein the stage of a disease or disorder in a subject is determined when the absence, presence or amount of analyte in step (e) is compared with the amount of analyte present in an analogous sample from a subject having no disease and/or disorder or having a particular stage of the disease or disorder. In a further embodiment, prior to step (a), the method comprises the step of attaching a first molecule to a solid support. In another embodiment, the binding molecules are contacted with the sample sequentially. In one embodiment, a step to remove unbound'sample or binding molecule may be performed at any step prior to step (e).

In another preferred embodiment, binding of the second molecule is detected or measured when the analyte is bound to the first molecule. In another preferred embodiment, binding of the second molecule is detected or measured when the analyte is bound, through the first molecule, to a solid support.

In an alternate embodiment, a step to remove unbound sample or binding molecule may be performed at any step prior to step (e). For example, several steps prior to step (e) can be introduced, in lieu of steps (b) and (d), to selectively remove unbound sample, unbound first molecules or unbound second molecules.

To provide a basis for the diagnosis or prognosis of a disease or disorder or response to treatment associated with an analyte, a normal or standard profile for expression is established, using various techniques known in the art. For example, a sample (e.g., body fluid, cell extract) taken from normal subjects, either animal or human, is contacted with a protein (e.g., antibody) capable of binding an analyte of interest in the sample, and under conditions suitable for association, is detected and measured. Values obtained from these normal subjects are compared with values obtained from a parallel experiment using known amounts of the analyte of interest to calculate a standard value. Values obtained from a sample from a patient who has, or is at risk for contracting, a disease or disorder, or who is receiving treatment or who has received treatment, can be compared to the standard value, and deviation from the standard value is used to determine the prognosis and/or diagnosis of a disease or disorder and/or response or lack of response to treatment.

A diagnosis or prognosis or response to treatment can be established for any disease having a characteristic analyte such as, without limitation, those diseases disclosed above. If the presence of a disease or disorder is established in a subject, and a treatment protocol initiated, the above-described assays can be repeated on a regular basis to determine whether the values obtained from samples of the subject are, over time, approximating or further deviating from values observed in samples from normal subjects. The results obtained from such assays can assess the efficacy of treatment over the treatment period.

5.3. Kits of the Invention

The invention also relates to kits comprising one or more binding molecules, protein arrays for identifying binding proteins, and/or reagents useful for detecting binding of a molecule to an analyte.

In one embodiment, a kit comprises (a) in a first container, a purified biomolecular analyte; (b) in a second container, a first molecule that binds the analyte; and (c) a solid support having a second, different molecule attached thereto, wherein the second molecule binds the analyte when the analyte is bound to the first molecule, and wherein at least one of the first or second molecules is a non-antibody protein derived from a species different from that of the analyte.

In another embodiment, a kit comprises (a) in a first container, a purified biomolecular analyte; (b) a solid support having a first molecule attached thereto, wherein the first molecule binds the analyte, and wherein the first molecule is a non-antibody protein derived from a species different from that of the analyte; and (c) in a second container, a second, different molecule that binds the first molecule when the first molecule is bound to the analyte.

The kits of the invention can further comprise a detection means to detect the first molecule when bound to the analyte such as, for example, a reagent useful for assaying binding of a molecule to an analyte. In another embodiment, the kit comprises a detection means to detect the second molecule when bound to the first molecule such as, for example, a reagent useful for assaying binding of an antibody to another, different molecule.

The kits of the invention can further comprise additional binding proteins that bind either to the first molecule or to the second molecule. In such an embodiment, the kit comprises a means to detect one or more of such binding proteins. Alternatively, in another embodiment, the kit comprises a means to detect one or more of such further binding proteins in addition to one of either the first or the second binding protein.

The kits of the invention can further comprise a multiplicity of binding proteins such that one or two binding proteins bind(s) to the analyte and the remainder of the binding protein each binds, directly or indirectly, either to one or the other of the first two binding proteins. In such an embodiment, the kit comprises a detection means to detect one or more of such binding proteins.

The kits of the invention can further comprise a chart obtained from a protein interaction database which lists the interactions of the binding proteins of the kit. This chart can be assembled using information from a database of protein interactions for the protein(s) of interest.

One of the molecules that binds the analyte or that binds another molecule when bound to the analyte can be attached to the surface of a flat solid support, contained in wells on a solid support, or attached to the surface of wells on the solid support. In one embodiment, one of the molecules that binds the analyte or that binds another molecule when bound to the analyte is already attached to the solid support. In another embodiment, a molecule that binds the analyte or that binds another molecule when bound to the analyte is not attached to the wells of the solid support, but is contained in the wells. In yet another embodiment, a molecule that binds the analyte or that binds another molecule when bound to the analyte is not attached to the wells of the solid support, but is aliquoted in one or more containers, and can be added to the wells of the solid support. In one embodiment, the kit provides a substratum (e.g., beads) to which a molecule that binds the analyte or that binds another molecule when bound to the analyte, can be attached, after which the substratum with attached molecules can be placed into wells of the solid support.

In another embodiment, a kit comprises (a) in a first container, a population of proteins, optionally arrayed on a solid support; and (b) in a second container, a detection means for an analyte of interest, such that the analyte, after being tested for binding to the population of proteins-in the second container, is detected by the detection means, thus identifying proteins in the first container that might be suitable as reagents in an assay to measure the analyte.

In yet another embodiment, a kit comprises (a) in a first container, a reagent for conjugating a detection molecule to an analyte of interest; and (b) in a second container, a population of proteins, optionally arrayed on a solid support, such that the analyte, after being conjugated to the detection molecule, is tested for binding to the population of proteins in the second container.

### 6. EXAMPLE 1

### Assays

6.1. Assay for a Prostate Cancer Marker

Several markers that correlate with prostate cancer are known in the [symbol] such as, for example, prostate-specific antigen (PSA), human kallikrein-2, BPSA, pro-PSA, prostate-specific membrane antigen (PSMA), hepsin (a transmembrane serine protease), pim-1 (a serine/threonine kinase) (See, e.g., Dhanasekaran et al., 2001, “Delineation of prognostic biomarkers in prostate cancer”, Nature. 412:822-826). Such markers can be useful for the diagnosis, prognosis, staging, response to treatment and/or management of prostate cancer. PSA (also known as human glandular kallikrein 3), a kallikrein-like serine protease, is recognized as a valuable tumor marker for the screening, diagnosis and management of human prostate cancer. Levels of serum PSA levels have clinical significance in prostate disease management, such as evaluating risk for prostate cancer, determining pretreatment staging, monitoring treatment efficacy and detecting recurrence of disease (Gao et al., 1997, “Diagnostic and prognostic markers for human prostate cancer”, Prostate. 31 (4):264-281). In the following non-limiting example, the analyte of interest, for exemplary purposes, is human prostate specific antigen (“PSA”). Since PSA is relatively specific to the prostate gland, it is a good example of an analyte that does not have obvious homologs or orthologs in the species from which the binding protein reagents will be obtained (yeast).

PSA-binding partners are identified by screening an array of yeast proteins, all fused to GST and MisX6 at their N-termini, with PSA. Generally, binding of PSA to proteins on the yeast protein chip can be assayed as follows. Blocked protein chips are washed three to five times in PBS buffer, and the extra liquid on the glass surface is removed by tapping the slides vertically on a Kimwipe™. Biotinylated PSA (200 μl) is added to the protein chip and immediately covered by a hydrophobic plastic coverslip (Grace Bio-Labs, USA). After trapped air bubbles are removed, the chip is incubated in a humidity chamber at RT for one hour. The coverslip is removed by immersion in a large volume of PBS buffer (>50 ml). The chip is then moved to a second PBS bath (>50 ml) and washed 3×5 min with shaking at room temperature (RT). After removing excess liquid on the chip surface, at least 150 μl of Cy3-conjugated or Cy5-conjugated streptavidin (Pierce, USA; 1:2000 to 1:4000 dilution) is added to the chip surface and covered by a hydrophobic plastic coverslip (Grace Bio-Labs, USA). The chip is incubated for at least 30 min in the dark at RT. The chip is then washed as described above. To completely remove the liquid on the chip, the chip is spun to dryness at 1500-2000 rpm for 5-10 min at RT.

If a protein chip is to be screened with anti-biotin antibodies rather than streptavidin, the protein-antibody interaction can be detected as follows. Blocked protein chips are washed three to five times in PBS buffer, and the extra liquid on the glass surface is removed by tapping the slides vertically on a Kimwipe™. A primary antibody (200 μl properly diluted in PBS containing 1-3% BSA and 0.1% TritonX-100) is added to the protein chip and immediately covered by a hydrophobic plastic coverslip (Grace Bio-Labs, USA). After trapped air bubbles are removed, the chip is incubated in a humidity chamber at RT for one hour. The coverslip is removed by immersion in a large volume of PBS buffer (>50 ml). The chip is then moved to a second PBS bath (>50 ml) and washed 3×5 min with shaking at RT. After removing excess liquid on the chip surface, at least 150 μl of Cy3-conjugated or Cy5-conjugated secondary antibodies (properly diluted in PBS containing 1-3% BSA and 0.1% TritonX-100) is added to the surface and covered by a hydrophobic plastic coverslip (Grace Bio-Labs, USA). The chip is incubated for at least 30 min in the dark at RT. The chip is then washed as described above. To completely remove the liquid on the chip, the chip is spun to dryness at 1500-2000 rpm for 5-10 min at RT.

After binding proteins for PSA are identified, the respective yeast clones encoding the binding proteins (with N-terminal GST-HisX6 that can optionally be removed before use by selective proteolysis) are expressed in yeast under the control of a galactose-inducible GAL1 promoter. Yeast glycerol stock containing the clones for the binding proteins are inoculated into URA-/raffinose liquid media. After the culture reaches O.D.₆₀₀ of 4.0 in about 16 hours at 30° C. with vigorous shaking, one ml of the culture is inoculated into 100 μl of URA⁻/raffinose media. The cells are grown at 30° C. with vigorous shaking. After 12-16 hours of growth, the culture should reach O.D.₆₀₀ of 0.6 to 0.8. The cultures are discarded if the O.D.₆₀₀ is over 1.0. A 40% galactose stock is added to a fmal concentration of 2% to induce the cells. The cultures are induced at 30° C. for 4 hours with shaking. The cells are harvested by spinning at 3000 rpm for 2-10 min, and the cell pellets are resuspended in 100-1000 μl of cold water by vortexing. Cells are collected by centrifugation and resuspended in 100-1500 μl of cold lysis buffer containing Protease Inhibitors Cocktail with EDTA from Roche and PMSF, on ice. The washed cells are collected by a brief centrifugation, and the lysis buffer is discarded. The washed semi-dry culture is immediately stored in −80° C freezer.

The cells are lysed with glass beads. Glass beads (1 ml) are added to the frozen cell pellet. The cells are vortexed at high speed in the presence of protease inhibitors to lyse the cells. The required amount of glutathione beads (roughly 10-50 μl of beads per sample) (Amersham Pharmacia Biotech, USA) is washed four times with cold lysis buffer without the protease inhibitors, and then resuspended in 5× its original volume with lysis buffer containing fresh protease inhibitors. Glutathione beads are added to the cell lysate and incubated on a roller drum at 4° C. for one hour. The beads are collected by spinning at 3000 rpm for 10-60 seconds, and the [symbol] are washed once with 200-800 μl of a buffer containing protease inhibitors, and twice with buffer without the inhibitors. The beads are then washed three times with 200-800 μl of buffer. After complete removal of the buffer, 20-50 μl of low salt buffer is added to each well and incubated for 1 hour at 4° C. The eluate/beads slurry is transferred to a 96-well microtiter plate coated with glutathione. After binding for 1 hour, the plates are washed with PBS.

The binder partners remain bound to the glutathione-coated wells. The binder partners can be purified from the wells and used for a PSA assay.

A yeast-derived protein that binds PSA is attached to wells of a 96-well plate. Serum samples are obtained from men after radical prostate surgery. Serum samples (200 μl) and a positive and negative control are added to different wells of a 96-well plate. The plate is incubated for 2 hours at RT to allow for binding of the PSA in the sample to the yeast protein on the surface of the well. Each well of the plate is washed with 200-800 μl of phosphate-buffered saline and 0.1% Tween-20 to remove contaminating non-specific binding. The bound PSA is contacted with an anti-human PSA mouse monoclonal antibody conjugated to horseradish peroxidase and incubated for two hours to allow PSA to be bound by the antibody. Excess antibody is removed by washing. Binding of the antibody to PSA is detected by adding to each well a substrate for horseradish peroxidase, and the colorimetric reaction is measured in a 96-well plate reader. Optionally, the amount of binding is measured to estimate the circulating concentration of PSA in the patient. If screening of the yeast protein array reveals at least two proteins that bind PSA and if at least two of the proteins can bind PSA in the presence of the other, a non-antibody protein that binds PSA and that is conjugated to horseradish peroxidase can be substituted for the anti-PSA mouse monoclonal antibody. Since PSA recurrence after radical prostatectomy usually indicates recurrent prostate cancer, a qualitative assay to determine the presence or absence of serum PSA in men having received radical prostatectomy has clinical benefit. Values over background are considered positive for PSA, indicating an increased risk for prostate cancer.

6.2. Assay for a Marker of Human Immunodeficiency Virus

An assay is performed using two binding proteins derived from yeast to determine the presence of antibodies to the env13 protein of HIV which indicates exposure to HIV and risk of HIV infection resulting in acquired immunodeficiency syndrome (AIDS).

Proteins that bind to env13 protein of HIV are identified and isolated by screening a yeast protein [symbol] according the methods disclosed in Section 6.1. Two such yeast binding proteins are selected such that each can bind to the env13 protein in the presence of the other. The two proteins so identified in the above screen are prepared for use in the present diagnostic assay by being conjugated to horseradish peroxidase.

Wells of polystyrene microtiter plates are coated by passive adsorption with a mouse anti-human immunoglobulin antibody (a mixture of anti-huIgG and anti-huIgM, is used to detect circulating IgM and IgG antibodies specified against env13), and the plates are then washed. Serum samples are added to the coated well, incubated for a sufficient time and under conditions to allow anti-env13 antibodies in the sample to be bound by the anti-immunoglobulin antibodies that coat the wells, and the plates are again washed. Anti-env13 antibody, originally present in the sample, is now bound to the antibodies on the well surface.

Recombinant env13 protein is then added to the wells, and incubated for a sufficient time such that any anti-env13 antibody present can bind the env13 protein. Excess env13 protein is removed by washing. The two yeast binding proteins conjugated to horseradish peroxidase are then added to the wells. The presence of env13 protein, which is in turn indicative of the presence of anti-env13 antibody in the serum sample, is detected by adding a substrate of horseradish peroxidase, after which the colorimetric reaction is measured in an automated assay analyzer. Addition of two yeast reporter proteins that bind to different sites on env13 protein increase the chance of detecting any env-13 bound and serve to amplify the signal. Values over background are considered positive for the anti-env13 antibody, indicating-prior exposure of the patient to HIV and an increased risk for developing AIDS.

6.3. Assay for a Breast Cancer Marker

The HER2 proto-oncogene product is overexpressed in 30% of breast cancers, and correlates with poor prognosis. However, overexpression of HER2 proto-oncogene increases the probability of favorable response to therapeutic regimens including monoclonal antibodies specified against the proto-oncogene. A binding assay is performed to determine the level of the Her-2/neu (“ERBB2/c-erbB-2”) gene sequence, the amplification of which provides an indication of aggressive breast cancer. ERBB2/c-erbB-2 is frequently amplified in many human mammary tumors and in cell lines derived from such tumors (Kraus et al., 1987, EMBO J. 6:605-610). Thus, detection of an increased level of ERBB2/c-erbB-2 in a sample would indicate an increased risk of breast cancer.

The analyte measured in the present assay is the ERBB2/c-erbB-2 gene sequence. One binding [symbol] binding factor, a DNA-binding protein which binds to the promoter region of ERBB2/c-erbB-2 (Scott et al., 2000, “Ets regulation of the erbB2 promoter”, Oncogene. 19(55):6490-6502). A second binding protein is identified and isolated by screening a yeast protein array using methods described in Section 6.1. Briefly, a yeast protein chip is incubated with a solution containing an oligonucleotide comprising the sequence of the ERBB2/c-erbB-2 promoter. All binding proteins detected are then retested in the presence of Her-2/neu promoter binding factor such that the concentration of the latter protein is in excess of the concentration of the ERBB2/c-erbB-2 oligonucleotide. A yeast protein that retains the ability to bind the oligonucleotide despite the presence of the Her-2/neu promoter binding factor is selected as a suitable reagent for the assay. Preferably such yeast binding protein is also demonstrated to have a preference for binding ERBB2/c-erbB-2 oligonucleotides versus oligonucleotides with non ERBB2/c-erbB-2 sequences. The selected binding protein is detected, identified and isolated. Large-scale quantities of the selected binding protein are purified from the yeast strain containing the corresponding clone in accordance with the methods described in Section 6.1.

The wells of a 96-well microtiter plate are coated with Her-2/neu promoter binding factor. Tumor biopsy samples are placed in the wells and incubated to allow for binding of the ERBB2/c-erbB-2 promoter sequence to the Her-2/neu promoter binding factor. After overnight incubation allowing for binding to the ERBB2/c-erbB-2 gene, the plate is washed with a buffer containing 10 mM Tris-HCl, pH 7.5, 40 mM NaCl, 1 mM DTT, 1 mM EDTA. The wells are then incubated with the yeast binding protein, which is conjugated to a fluorescent tag. After washing with the same buffer, fluorescence is measured with a scanner. Fluorescence intensity indicates presence and amount of the ERBB2/c-erbB-2 gene sequence. Higher fluorescent signal, as compared with that of a control sample (a breast cancer sample in which it is known that the ERBB2/c-erbB-2 gene has not been amplified) containing the same amount of DNA, indicates amplification of the ERBB2/c-erbB-2 gene, which predicts an increased risk of breast cancer.

In an alternative embodiment of this assay, the Her-2/neu promoter binding factor is screened against a yeast protein chip and yeast proteins are identified that bind to this protein. These yeast proteins are tested to identify proteins that can bind simultaneously to Her-2/neu promoter binding factor while the Her-2/neu promoter binding factor binds its cognate DNA sequence. The latter proteins and the Her-2/neu promoter binding factor can all be conjugated to the same fluor. In the assay, after addition of the fluor-tagged Her-2/neu promoter binding factor, the sample can be washed once again and the fluor-tagged yeast proteins can be added to the [symbol] be made. In this way, the fluor signal can be amplified proportionately with the concentration of ERBB2/c-erbB-2 gene sequence, thus facilitating measurement of the concentration of the gene.

In yet another embodiment of this assay, amplification is achieved by the use in the assay of a first fluor-tagged yeast protein that binds the fluor-tagged Her-2/neu promoter binding factor and a second fluor-tagged yeast protein that binds the first fluor-tagged yeast protein.

6.4. Assay for Hepatitis B Surface Antigen

The hepatitis B surface antigen (“HBsAg”) is a component of the external envelope of the hepatitis B virus (“HBV”) particle (Gerlich, 1993, Viral Hepatitis, Churchill Livingstone (ed), pp. 83-113). The detection of HBsAg in human serum or plasma indicates an infection by the hepatitis B virus. HbsAg is the first immunological marker detectable in the bloodstream, and is generally present several days or weeks before clinical symptoms begin to appear in the infected individual. HbsAg is observed in the blood of persons with acute and chronic HBV infections. HBsAg screening assays are used to identify persons infected with HBV. Identification of infected persons can, among other things, help prevent transmission of HBV via blood and blood products. HBsAg assays are also used to monitor the course of the disease in persons with acute or chronic HBV infections. In addition, tests for the presence of HBsAg are recommended as part of prenatal care to take steps to prevent HBV transmission to a newborn child.

An assay is performed to detect HbsAg in human serum. The assay uses two yeast proteins that bind to HbsAg. Proteins that bind HbsAg are identified by screening a yeast protein chip as described in Section 6.1. A complex prepared between HbsAg and one of the selected proteins is then tested against the other HbsAg binding proteins to identify pairs of yeast binding proteins such that binding of a first protein to HbsAg does not interfere with the binding of a second protein to HbsAg. The assay, based on a sandwich assay principle, is carried out on a Roche Elecsys 1010 immunoassay analyzer. A first HbsAg-binding-protein is conjugated to biotin. A second HbsAg-binding-protein is conjugated to an electrochemiluminescent ruthenium complex. In the first incubation, 50 μl of serum sample is contacted with both the first and second binding proteins to form a sandwich. Magnetic microparticles coated with streptavidin are added to the reaction and incubated for 10 minutes. The complex, containing the biotinylated binding protein, is sound to the solid phase via interaction of biotin and streptavidin. The reaction mixture is aspirated into a measuring cell where the microparticles are magnetically captured onto the surface of an electrode. Application of voltage to the electrode induces chemiluminescent emission which is measured by a photomultiplier. Results are calculated automatically by the Elecsys software by comparing the electrochemiluminescence signal from the sample with a previously determined threshold value.

6.5. Assay for Human Chorionic Gonadotrophin

An assay is performed in accordance with the methods of the invention to detect human chorionic gonadotrophin (hCG), a marker for pregnancy. hCG appears in the blood and urine of pregnant women approximately 6-7 days after conception.

The assay uses two binding proteins that bind hCG, which are identified by screening a yeast protein array as described in Section 6.1. The assay is conducted on a membrane strip having a first and second binding protein, each that bind to hCG, attached to the strip, such that the second binding protein is permanently attached to the strip. The first binding protein, which is calorimetrically labeled, is attached to a portion of the membrane strip to which the sample is added. A urine sample is contacted with the portion of the membrane strip to which the first binding protein is attached. The urine hydrates the first binding protein, thereby allowing hCG in the sample to be bound by the first binding protein. The hCG-first binding protein complex (“complex”) then diffuses along the membrane chromatographically, ultimately reaching the portion of the membrane strip to which the second binding protein is permanently attached. The complex binds to the second binding protein, thereby concentrating the colorimetrically labeled first binding protein resulting in the appearance of a colored line visible to the naked eye.

The appearance of the line indicates a positive result for hCG. If no line appears, the result is negative. As a control, unbound first binding protein diffuses further along the membrane strip and contacts a compound that binds and concentrates the first binding protein, resulting in the appearance of a colored line and thus confirming that the assay was correctly conducted.

### 7. EXAMPLE 2

### Detection of a Human Protein Analyte (RAS) Using a Yeast Proteome Microarray

A yeast proteome microarray containing nearly all yeast proteins was prepared and screened for a number of biochemical activities. A high-quality collection of 5800 yeast ORFs (93.5% of the total) was cloned into a yeast high-copy expression vector using [symbol] Yeast 9:715). The yeast proteins were fused to GST-HisX6 at their amino termini and expressed in yeast under the control of a galactose-inducible GAL1 promoter (Zhu et al., 2000, Nature Genet. 26:283-289; Mitchell et al., 1993, 9(7):715-722). The yeast expression strains contain individual plasmids in which the correct yeast ORFs have been shown to be properly fused in-frame to GST by DNA sequencing.

7.1. Materials and Methods

Briefly, yeast ORFs were amplified by PCR and co-transformed into yeast cells along with the vector to generate expression clones. The plasmids were rescued in E. coli, and the vector-insert junctions were sequenced. If the ORF cloned was not the ORF of interest, or a frameshift was detected, the cloning cycle was repeated. Once a construct was confirmed, the plasmid DNA was reintroduced into yeast and E. coli to create permanent stocks for future analyses (Zhu et al., 2000, Nature Genet. 26:283-289). By repeating the cloning cycle, 5800 unique yeast ORFs were successfully cloned, representing 93.5% of the total.

To generate purified proteins for biochemical analysis, a robust and high-throughput purification method for preparing proteins in a 96-well format was developed and optimized. Using glutathione-agarose beads, yeast extracts were prepared, and fusion proteins were purified. The lysis buffer and initial washes contained 0.1% Triton to ensure that the purified proteins were free of lipids. Using the methods of the invention as disclosed herein, at least 1152 protein samples can be prepared from cells in under 10 hours. The quality and quantity of the purified proteins were monitored using immunoblot analysis of 60 random samples. Greater than 80% of the strains produced detectable amounts of fusion proteins of the expected molecular weight.

7.1.1. Yeast Culture Preparation

The following steps were carried out in the following order:

1. Yeast glycerol stocks stored in 96-well plates at −80° C. were inoculated onto a URA-agar plate (Omni, USA) using a 96-pronger.

2. The culture was allowed to grow on agar at 30° C. for 48 hours, until the time at which visible colonies (2 mm diameter) were observed.

3. A 96-pronger was used to inoculate yeast cells from agar plates to a 96-well 2 ml box in which every well contained URA-/raffinose liquid media and a 2 mm diameter glass ball, which faciliates the uniform growth [symbol] .

4. After the culture reached O.D600 4.0 in about 16 hours at 30° C. with vigorous shaking (300 rpm), 15 μl of the same strain was inoculated into 750 μl of URA-/raffinose liquid media in four different boxes to obtain 3 ml of culture. Again, each well contained the same glass ball to achieve aeration and even growth. The cells were grown at 30° C. with vigorous shaking.

5. After 12-16 hours of growth, the culture should reach O.D.600 0.6 to 0.8. Using an automated liquid-handling device (Q-Fill, Genetix, UK), 40% galactose stock was added to each well to a final concentration of 2% to induce the cells. The cultures were induced at 30° C. for 4 hours with shaking.

6. The cells were harvested by spinning at 3000 rpm for 2-10 min, and the cell pellets were resuspended in cold water by vortexing. Cells of the same strain were then merged from 4 wells into one. Cells were collected by spinning and resuspended in cold lysis buffer, without the protease inhibitors, on ice. The washed cells were collected by a brief centrifugation, and the lysis buffer was discarded. The washed semi-dry culture was immediately stored in −80° C. freezer. The culture can be kept for weeks.

7.1.2. Protein Purification in a 96-Well Fashion

The following steps were carried out in the following order:

1. The frozen culture in a 96-well box was transferred from −80° C. to ice and 100-300 microliter of zirconia beads (0.5 mm diameter from BSP, Germany) was added to 25 each well. While the culture was still frozen, lysis buffer containing fresh protease inhibitors was added. A cap mat was used to seal each well. After thawing the culture for 5-25 min on ice, the cells in the 96-well box were vortexed 20-60 seconds for 3-6 times with 1-5 min intervals on ice. To efficiently disrupt the yeast cell wall, and to process many plates at once, a paint shaker (HARBIL™ 5G HD, 36 kg capacity, adjustable pressure and shaking time, fixed speed at 200 times per minute) was used to violently agitate the samples.

Lysis Buffer:

30-300 mM Tris pH 7.5
50-300 mM NaCl
0.1-10 mM EGTA
0.01-1.0% TritonX-100
0.01-1% beta-mercaptoethanol
0.1-3 mM phenylmethylsulfonyl fluoride (“PMSF”)

2. After spinning at 3000 rpm for 2-10 min. the supernatant was collected using wide-open tips (Fisher, USA) and transferred into a 96-well filter plate (Whatman, USA; Whatman UNIFILTER™, Cat. No. 7700-1801 having a hydrophilic PVDF filter), which was placed on top of a 96-well box.

3. To obtain more proteins, 100-500 μl of lysis buffer containing fresh protease inhibitors was added to the cell debris, and Steps 1 and 2 were repeated.

4. The combined cell lysate was spun through the filter plate into a cold and clean 96-well box for 10-30 min at 3000 rpm.

5. Meanwhile, the required amount of glutathione beads (Amersham, USA) was washed four times with cold lysis buffer without the protease inhibitors, and finally resuspended in 5× of its original volume with lysis buffer containing fresh protease inhibitors.

6. 100 μl of washed glutathione beads was added to each well and sealed tightly with a cap mat. The beads were incubated with the lysate on a roller drum at 4° C. for one hour. To obtain the best mixing, the boxes were rotated 360 degrees on the roller drum.

7. The beads were collected by spinning at 3000 rpm for 10-60 seconds, and the supernatant was discarded. Beads were washed once with wash buffer containing protease inhibitors, and twice without the inhibitors.

Wash Buffer:

30-300 mM Tris or 50-200 mM HEPES pH 7.5
50-600 mM NaCl
0.0-10 mM EGTA
0.0-1.0% TritonX-100
0.01-1% beta-mercaptoethanol (“BME”)
0.1-3 mM PMSF
0-15% glycerol
Roche Protease inhibitor tablets (containing EDTA)

8. The beads were then washed three times with wash buffer. After complete removal of the buffer, 20-50 microliters of elution buffer was added to each well. Filter plates used for the elution step comprised materials having low affinity for proteins [symbol] .

The box was vortexed briefly to resuspend the beads and incubated on a roll drum for one hour at 4° C.

Elution Buffer:

50-200 mM HEPES pH 7.5
50-200 mM NaCl
20-40% Glycerol
5-40 mM Glutathione (Reduced form)

9. The eluate/beads slurry was transferred to a cold filter plate (Millipore, USA), and the eluate was collected to a 96-well PCR plate by spinning through the filter plate for 0.5-2 min at 3000 rpm at 4° C.

10. Each purified protein was aliquoted into three 96-well PCR plates and immediately stored in a −80° C. freezer.

7.1.3. Method of Making a Proteome Microarray

To prepare the proteome chips, 5800 different yeast proteins were printed in duplicate onto nitrocellulose-coated glass slides (FAST™ slides, Schleicher & Schueli, Keene, N.H.) using a commercially available microarrayer. Various controls, including Cy5-labeled BSA, biotinylated IgG, and dilutions of GST, were also printed.

To determine how much fusion protein was attached to the surface of the slide, and to assess the reproducibility of the protein attachment, chips were probed with anti-GST antibodies. Over 93.5% of the protein samples gave signals significantly above background (i.e., greater than approximately 10 fg of protein). A comparison with known amounts of GST also printed on the slide, indicated that about 90% of the spots contain approximately 10 fg to 950 fg of protein. Detection of proteins on a proteome chip with fluorescently labeled antibodies is extremely sensitive, i.e., the signal-to-noise ratio is high despite that only 1/10,000 of purified proteins from a 3-ml culture is spotted on the slide. To test the reproducibility of the protein spotting, the signals from each pair of duplicated spots were compared with one another, and 95% of the signals were within 5% of the average.

7.1.4. Method of Using a Proteome Microarray

Proteome chips were tested by probing for several exemplary types of biological activities: protein-protein interactions, protein-nucleic acid interactions, and protein-lipid [symbol] chips were blocked by slowly immersing the printed glass slides into either BSA (1-3% (w/w) BSA in PBS buffer; SIGMA™, USA) or glycine blocking buffer (30-300 mM glycine; 50-300 mM Tris, pH 6.5-8.5; 50-300 mM NaCl; SIGMA™, USA) with the protein side up. The buffer was filtered through a 2 micron filter unit to remove particles. The slides were incubated in the blocking buffer at 4° C. overnight without any shaking (disturbance of the blocking buffer may result in the protein streaks on the glass surface).

Probe proteins were generally prepared as follows. Yeast proteins were purified by affinity column using glutathione beads from 50 ml culture using standard protocols without the elution from the beads. The protein beads were washed three to five tines with cold PBS buffer (pH 8.0) (SIGMA™, USA). Approximately 1 ml of Sulfo-NHS-LC-LC-Biotin (PIERCE™ Cat. No. 21338, USA) dissolved in PBS (pH 8.0) at a concentration of 0.1-50 mg/ml was added to the glutathione beads and incubated at 4° C. for 2 hours. The beads were washed 5 times with cold PBS buffer (pH 8.0) and eluted with 100-500 microliter of the elution buffer (50-200 mM HEPES pH 7.5; 50-200 mM NaCl; 20-40% glycerol; 5-40 mM glutathione). Protocols resulting in more weakly biotinylated proteins are preferred. Batches of proteins that are biotinylated to different degrees were pooled for future usage.

7.2. Results and Discussion

7.2.1. Identification of Human Ras-Interacting Yeast Proteins

To demonstrate the use of non-antibody proteins from one species to detect and measure a protein analyte from another species, the yeast proteome microarray constructed as described above was probed with the human protein ras. Ras genes are evolutionarily conserved and codify for a monomeric G protein binding GTP (active form) or GDP (inactive form) (Macaluso et al., 2002, Ras family genes: an interesting link between cell cycle and cancer, J Cell Physiol. 192(2):125-30). Mutations in each ras gene frequently are found in different tumors, suggesting their involvement in the development of specific neoplasia. These mutations lead to a constitutively active and potentially oncogenic protein that could cause a deregulation of cell cycle. Recent observations have begun to clarify the complex relationship between Ras activation, apoptosis, and cellular proliferation. A greater understanding of these processes would help to identify the factors directly responsible for cell cycle deregulation in several tumors, moreover it would help the design of specific therapeutic strategies, for the control on the proliferation of neoplastic cells. In this example, a human ras protein probe was biotinylated, and bound probe was detected using Cy3-labeled streptavidin. The yeast proteome was also probed with the homologous yeast has [symbol] protein.

Briefly, recombinant GST-fusions of the human and yeast ras proteins were purified and biotinylated as described above. Yeast proteome slides were blocked with 50 ml 1× PBS, 0.1% Tween-20, 1% BSA for one hour in the cold room with shaking. 200 μl (approximately 10 μg) of biotinylated human or yeast ras proteins were added to separate arrays. Probings were carried out on ice for 1 hour, then washed with 1× PBS, 0.1% Tween-20 (PBST). Three μl of Cy5-Streptavidin (PIERCE, USA; 1:2000 to 1:4000 dilution) in 50 ml of PBST+0.3% BSA were then added and incubated for one hour at 6° C. with shaking. Slides were then washed with PBST, rinsed twice with dH₂O and then centrifuged (4000 rpm) dry for 1 minute). Slides were scanned on an Axon Instruments microarray scanner at 100% laser power and 500 PMT settings.

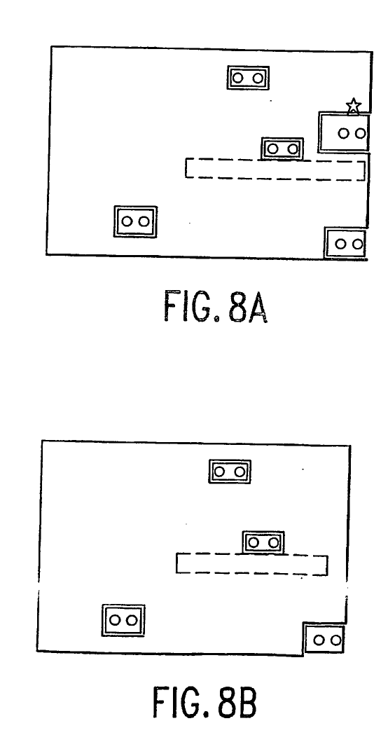

The results of probing the yeast proteome microarrays with human and yeast ras proteins are shown in FIG. 8. The left panel (A) shows a portion of the scanned image from the yeast proteome microarray that was probed with the human ras protein. The right panel (B) shows a portion of the scanned image from the yeast proteome microarray that was probed with the yeast ras protein. Solid white boxes are drawn around pairs of spots representing a single protein that interacts specifically with the probe. A dashed white box is drawn around control spots. It can be seen in this figure that four proteins interact with both the human and yeast ras proteins. This is not surprising since there is known to be a significant degree of homology between the ras proteins of the two species. It should also be noted that one yeast protein (designated with a star in the left panel) only interacts specifically with the human protein. This protein, therefore, can be used as an affinity reagent to specifically detect the human ras protein.

### 8. REFERENCES CITED

All references cited herein are incorporated herein by reference in their entirety and for all purposes to the same extent as if each individual publication or patent or patent application was specifically and individually indicated to be incorporated by reference in its entirety for all purposes.

### 9. EQUIVALENTS

Many modifications and variations of this invention can be made without departing from its spirit and scope. A person of ordinary skill in the art will recognize, or be able to ascertain through routine experimentation, various alternatives, adaptations, and modifications to the particular embodiments of the invention described herein, all of which [symbol] the claimed invention intends to encompass all such equivalents. Thus, the specific embodiments described herein are offered by way of example only, and the invention is to be limited only by the terms of the appended claims, along with the full scope of equivalents to which such claims are entitled.

1. A method for detecting or measuring an analyte comprising the steps of:
(a) contacting a first molecule that binds a biomolecular analyte with a sample containing said analyte under conditions that allow said analyte to be bound by said first molecule;
(b) contacting said bound analyte with a second, different molecule that binds said analyte when said analyte is bound to said first molecule, under conditions that allow said analyte to be bound by said second molecule;
(c) detecting or measuring binding of said second molecule to said analyte when said analyte is bound to said first molecule;
wherein at least one of said first and second molecules is a non-antibody protein that is derived from a species different from that of said analyte; wherein said first molecule is attached to a solid support either before or after step (a); and wherein detection or measurement of binding indicates presence or amount, respectively, of said analyte.

2. A method for detecting or measuring an analyte comprising the steps of:
(a) contacting a first molecule that binds a biomolecular analyte with a sample containing said analyte under conditions that allow said analyte to be bound by said first molecule;
(b) contacting said bound, first molecule with a second, different molecule that binds said first molecule when said first molecule is bound to said analyte, under conditions that allow said second molecule to be bound by said first molecule;
and
(c) detecting or measuring binding of said second molecule to said first molecule when said analyte is bound to said first molecule;
wherein said first molecule is a non-antibody protein that is derived from a species different from that of said analyte; wherein said first molecule is attached to a solid support [symbol] presence or amount, respectively, of said analyte.

3. The method of claim 1 or claim 2, which further comprises, prior to step (c), the step of removing unbound sample.

4. The method of claim 1 or claim 2, which further comprises, prior to step (c), the step of removing unbound second molecule.

5. The method of claim 1 or claim 2, wherein said first and second molecules are non-antibody proteins.

6. The method of claim 1, wherein said first and second molecules are derived from a species different from that of said analyte.

7. The method of claim 6, wherein said first and second molecules are derived from the same species.

8. The method of claim 6, wherein said first and second molecules are derived from different species.

9. The method of claim 1 or claim 2, further comprising, prior to step (a), the step of attaching said first molecule to said solid support.

10. The method of claim 1 or claim 2, wherein at least one of said first and second molecules is derived from yeast.

11. The method of claim 1 or claim 2, wherein said analyte is human-derived, and wherein said first molecule or second molecule is derived from yeast.

12. The method of claim 1 or claim 2, wherein said molecule that binds said analyte is identified by a method comprising the steps of:
(a) contacting said analyte with a positionally addressable array comprising a plurality of proteins, with each protein being at a different position on a solid support; and
(b) [symbol] ;
wherein the plurality of proteins comprises at least one protein encoded by at least 50% of the known genes in a single species; and wherein detection of said interaction at a position on said solid support identifies a molecule that binds said analyte.

13. The method of claim 1 or claim 2, wherein said molecule that binds said analyte is identified by a method comprising the steps of:
(a) contacting said analyte with a positionally addressable array comprising a plurality of proteins, with each protein being at a different position on a solid support; and
(b) detecting any analyte-protein interaction;
wherein the plurality of proteins comprises at least 50% of all proteins expressed in a single species, wherein protein isoforms and splice variants are counted as a single protein; and wherein detection of said interaction at a position on said solid support identifies a molecule that binds said analyte.

14. The method of claim 1 or claim 2, wherein said molecule that binds said analyte is identified by a method comprising the steps of:
(a) contacting said analyte with a positionally addressable array comprising a plurality of proteins, with each protein being at a different position on a solid support; and
(b) detecting any analyte-protein interaction;
wherein the plurality of proteins comprises at least 1000 proteins expressed in a single species; and wherein detection of said interaction at a position on said solid support identifies a molecule that binds said analyte.

15. The method of claim 1 or claim 2, wherein molecule that binds said analyte is identified by a method comprising the steps of:
(a) contacting said analyte with a positionally addressable array comprising a plurality of proteins, with each protein being at a different position on a solid support; and
(b) detecting any analyte-protein interaction;
wherein the plurality of proteins in aggregate comprise proteins encoded by at least 1000 different known genes in a single species; and wherein detection of said interaction at a position on said solid support identifies a molecule that binds said analyte.

16. The method of claim 1 or claim 2, wherein said detecting is performed by autoradiography, phosphoimager analysis, binding of hapten, immunofluorescence, [symbol] polymerase chain reaction, or colorimetric procedures.

17. The method of claim 1 or claim 2, wherein said analyte is a protein, lipid, nucleic acid, or small molecule.

18. The method of claim 1 or claim 2, wherein said analyte is a marker for a disease or disorder.

19. The method of claim 18, wherein said disease or disorder is an allergy, anxiety disorder, autoimmune disease, behavioral disorder, birth defect, blood disorder, bone disease, cancer, circulatory disease, tooth disease, depressive disorder, dissociative disorder, ear condition, eating disorder, eye condition, food allergy, food-borne illness, gastrointestinal disease, genetic disorder, heart disease, hormonal disorder, infectious disease, insect-transmitted disease, nutritional disorder, kidney disease, leukodystrophy, liver disease, mental health disorder, metabolic disease, mood disorder, neurological disorder, neurodegenerative disorder, personality disorder, phobia, pregnancy complication, prion disease, prostate disease, respiratory disease, sexual disorder, skin condition, sleep disorder, speech-language disorder, sports injury, tropical disease or vestibular disorder.

20. A method for diagnosing a disease or disorder in a subject comprising the steps of:
(a) contacting a first molecule that binds a biomolecular analyte with a sample, suspected of containing said analyte, from said subject under conditions that allow said analyte to be bound by said first molecule;
(b) contacting said bound analyte with a second, different molecule that binds said analyte when said analyte is bound to said first molecule, under conditions that allow said analyte to be bound by said second molecule; and
(c) detecting or measuring binding of said second molecule to said analyte when said analyte is bound to said first molecule, wherein detection or measurement of binding indicates presence or amount, respectively, of said analyte; wherein at least one of said first and second molecules is a non-antibody protein that is derived from a species different from that of said analyte; wherein said first molecule is attached to a solid support either before or after step (a); and wherein said disease or disorder is-determined to be present when the [symbol] amount of analyte present in an analogous sample from a subject not having said disease or disorder.

21. A method for diagnosing a disease or disorder in a subject comprising the steps of:
(a) contacting a first molecule that binds a biomolecular analyte with a sample, suspected of containing said analyte, from said subject under conditions that allow said analyte to be bound by said first molecule;
(b) contacting said bound, first molecule with a second, different molecule that binds said first molecule when said first molecule is bound to said analyte, under conditions that allow said first molecule to be bound by said second molecule; and
(c) detecting or measuring binding of said second molecule to said first molecule when said analyte is bound to said first molecule, wherein detection or measurement of binding indicates presence or amount, respectively, of said analyte; wherein said first molecule is a non-antibody protein that is derived from a species different from that of said analyte; wherein said first molecule is attached to a solid support either before or after step (a); and wherein said disease or disorder is determined to be present when the presence or amount of analyte in step (c) differs from a control value representing the amount of analyte present in an analogous sample from a subject not having said disease or disorder.

22. The method of claim 20 and claim 21, which further comprises, prior to step (c), the step of removing unbound sample.

23. The method of claim 20 and claim 21, which further comprises, prior to step (c), the step of removing unbound second molecule.

24. The method of claim 20 or claim 21, which further comprises, prior to step (a), the step of attaching said first molecule to said solid support.

25. The method of claim 20 or claim 21, wherein said disease or disorder is an allergy, anxiety disorder, autoimmune disease, behavioral disorder, birth defect, blood disorder, bone disease, cancer, circulatory disease, tooth disease, depressive disorder, dissociative disorder, ear condition, eating disorder, eye condition, food allergy, food-borne [symbol] infectious disease, insect-transmitted disease, nutritional disorder, kidney disease, leukodystrophy, liver disease, mental health disorder, metabolic disease, mood disorder, neurological disorder, neurodegenerative disorder, personality disorder, phobia, pregnancy complication, prion disease, prostate disease, respiratory disease, sexual disorder, skin condition, sleep disorder, speech-language disorder, sports injury, tropical disease, vestibular disorder prostate cancer, acquired immunodeficiency syndrome, hepatitis or breast cancer.

26. The method of claim 25, wherein said disorder is prostate cancer, acquired immunodeficiency syndrome, hepatitis or breast cancer.

27. A method for staging a disease or disorder in a subject comprising the steps of:
(a) contacting a first molecule that binds a biomolecular analyte with a sample, suspected of containing said analyte, from said subject under conditions that allow said analyte to be bound by said first molecule;
(b) contacting said bound analyte with a second, different molecule that binds said analyte when said analyte is bound to said first molecule, under conditions that allow said analyte to be bound by said second molecule; and
(c) detecting or measuring binding of said second molecule to said analyte when said analyte is bound to said first molecule, wherein detection or measurement of binding indicates presence or amount, respectively, of said analyte; wherein at least one of said first and second molecules is a non-antibody protein that is derived from a species different from that of said analyte; wherein said first molecule is attached to a solid support either before or after step (a); and wherein the stage of a disease or disorder in a subject is determined when the presence or amount of analyte in step (c) is compared with the amount of analyte present in an analogous sample from a subject having a particular stage of said disease or disorder.

28. A method for staging a disease or disorder in a subject comprising the steps of:
(a) contacting a first molecule that binds a biomolecular analyte with a sample, suspected of containing said analyte, from said subject under conditions that allow said analyte to be bound by said first molecule;
(b) contacting said bound, first molecule with a second, different molecule that binds said first molecule when said first molecule is bound to said analyte, under conditions that allow said first molecule to be bound by said second molecule; and
(c) detecting or measuring binding of said second molecule to said first molecule when said analyte is bound to said first molecule, wherein detection or measurement of binding indicates presence or amount, respectively, of said analyte; wherein said first molecule is a non-antibody protein that is derived from a species different from that of said analyte; wherein said first molecule is attached to a solid support either before or after step (a); and wherein the stage of a disease or disorder in a subject is determined when the presence or amount of analyte in step (c) is compared with the amount of analyte present in an analogous sample from a subject having a particular stage of said disease or disorder.

29. The method of claim 27 or claim 28, which further comprises, prior to step (c), the step of removing unbound sample.

30. The method of claim 27 or claim 28, which further comprises, prior to step (c), the step of removing unbound second molecule.

31. The method of claim 27 or claim 28, which further comprises, prior to step (a), the step of attaching said first molecule to said solid support.

32. The method of claim 27 or claim 28, wherein said disease or disorder is an allergy, anxiety disorder, autoimmune disease, behavioral disorder, birth defect, blood disorder, bone disease, cancer, circulatory disease, tooth disease, depressive disorder, dissociative disorder, ear condition, eating disorder, eye condition, food allergy, food-borne illness, gastrointestinal disease, genetic disorder, heart disease, hormonal disorder, infectious disease, insect-transmitted disease, nutritional disorder, kidney disease, leukodystrophy, liver disease, mental health disorder, metabolic disease, mood disorder, neurological disorder, neurodegenerative disorder, personality disorder, phobia, pregnancy complication, prion disease, prostate disease, respiratory disease, sexual disorder, skin condition, sleep disorder, speech-language disorder, sports injury, tropical disease, vestibular disorder prostate cancer, acquired immunodeficiency syndrome, hepatitis or breast cancer.

33. The method of claim [symbol] cancer, acquired immunodeficiency syndrome, hepatitis or breast cancer.

34. A kit comprising:
(a) in a first container, a purified biomolecular analyte;
(b) in a second container, a first molecule that binds said analyte; and
(c) a solid support having a second, different molecule attached thereto, wherein said second molecule binds said analyte when said analyte is bound to said first molecule, and wherein at least one of said first or second molecules is a non-antibody protein derived from a species different from that of said analyte.

35. A kit comprising:
(a) in a first container, a purified biomolecular analyte;
(b) a solid support having a first molecule attached thereto, wherein said first molecule binds said analyte, and wherein said first molecule is a non-antibody protein derived from a species different from that of said analyte; and
(c) in a second container, a second, different molecule that binds said first molecule when said first molecule is bound to said analyte.

36. The kit according to claim 34 or claim 35, further comprising a detection means to detect said first molecule when bound to said analyte.
